(19) United States
(12) Patent Application Publication
(10) Pub. No.: US 2026/0034208 A1
(43) Pub. Date: Feb. 5, 2026

(54) VIRUS-LIKE VESICLES (VLVS) BASED VACCINES AND METHODS OF PREVENTING, AMELIORATING, AND/OR TREATING COVID-19 AND/OR HEPATOCELLULAR CARCINOMA (HCC)

(71) Applicant: Yale University, New Haven, CT (US)

(72) Inventors: Chen Liu, New Haven, CT (US); Lei Yang, New Haven, CT (US); John Rose, New Haven, CT (US)

(21) Appl. No.: 18/997,279

(22) PCT Filed: Jul. 24, 2023
(86) PCT No.: PCT/US2023/070864

§ 371 Date: Jan. 21, 2025

Related U.S. Application Data
(60) Provisional application No. 63/392,320, filed on Jul. 26, 2022.
(60) Provisional application No. 63/392,351, filed on Jul. 26, 2022.

(51) Int. Cl. A61K 39/215; A61K 39/00; A61P 31/14; A61P 35/00; C12N 7/00; C12N 15/86

(52) U.S. Cl. CPC A61K 39/215; A61K 39/001174; A61P 31/14; A61P 35/00; C12N 7/00; C12N 15/86; A61K 2039/5258; A61K 2039/545; A61K 2039/55566; A61K 2039/55577; A61K 2039/575; C12N 2770/20034; C12N 2770/36122; C12N 2770/36123; C12N 2770/36134; C12N 2770/36152

(57) ABSTRACT

The present disclosure relates to the discovery of compositions and methods for therapeutic immunization for SARS-CoV-2 infections and/or disease(s) associated with expression of Glypican-3 (GPC3), including but not limited to cancers such as hepatocellular carcinoma (HCC). Methods of the disclosure include a method of generating virus like vesicles (VLVs), VLVs comprising SARS-CoV-2 antigens from a high titer VLV producing vector, VLVs comprising GPC3 antigens from a high titer VLV producing vector, methods of treating, ameliorating, and/or preventing SARS-COV-2 infection, methods of inducing a memory T and B cell immune response against SARS-CoV-2 infection in a, methods of treating, ameliorating, and/or preventing GPC3 associated disease, and methods of inducing a memory T and B cell immune response against GPC3 in a subject. Furthermore, the disclosure encompasses a pharmaceutical composition for vaccinating a subject to protect the subject against infection with

### CROSS-REFERENCE TO RELATED APPLICATION

This application claims priority to U.S. Provisional Patent Application No. 63/392,320 filed Jul. 26, 2022, and U.S. Provisional Patent Application No. 63/392,351 filed Jul. 26, 2022, the disclosures of which are incorporated herein by reference in their entirety.

### BACKGROUND

The 2019 coronavirus disease (COVID-19), which is caused by severe acute respiratory syndrome coronavirus 2 (SARS-CoV-2), has become an unprecedented pandemic that continues to cause significant morbidity and mortality globally. As of early 2022, SARS-CoV-2 has caused more than 484 million documented cases and nearly 6 million deaths worldwide. COVID's quick emergence and uncontrolled widespread have imposed a continuous threat to the physical and economic health of mankind. The introduction of mRNA and adenovirus based vaccines have helped lessen the severity of the disease for millions of people. However, viral mutation and the fading of induced immunity continue to present long-term challenges to controlling the pandemic. Developing additional vaccines with technologies that can offer advantages over mRNA and existing adenoviral vectors are needed to contain the spread of SARS-CoV-2.

Hepatocellular carcinoma (HCC) is the most common form of liver cancer and the leading cause of cancer-related mortality globally. In the United States, HCC is the ninth leading cause of cancer deaths. Despite recent clinical advances in prevention, screening, and new technologies in both diagnosis and treatment, incidence and mortality continue to rise. Factors that contribute to HCC include cirrhosis and hepatitis B and C infection. Alcohol consumption remains an important additional risk factor which contributes to liver damage that can lead to HCC. Current first-line treatment modalities include chemotherapy, radiation, and liver transplant. Despite recent advances in treatment strategies. HCC is an aggressive cancer that has a high risk of relapse and recurrence of poor prognosis. Recent advances in immunotherapy have demonstrated that the immune system can be an effective agent for the recognition and destruction of tumor cells and tissues. Thus, developing novel immunotherapy approaches to treat HCC is crucial to solving this global public health problem.

For these reasons, there remains a need in the art for effective SARS-CoV-2 vaccines for prophylactic and/or therapeutic use. Further, there remains a need in the art for effective vaccines that can be used to treat, ameliorating, and/or preventing HCC in the clinic. The present disclosure addresses these needs.

### BRIEF SUMMARY OF DISCLOSURE

In one aspect, the disclosure provides compositions comprising one or more virus like vesicles (VLVs) produced by one or more VLV producing vectors of the disclosure.

In one aspect, the disclosure provides a virus like vesicle (VLV)-producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a SARS-CoV-2 antigen or fragment thereof, which is operably linked to a 2A DNA encoding a 2A peptide.

In certain embodiments, the 2A DNA is operably linked to a vesicular stomatitis virus (VSV) G DNA encoding a VSV G protein. In certain embodiments, the SFV non-structural protein nucleotide sequences comprise at least two of the mutations selected from the group consisting of G-4700-A, A-5424-G, G-5434-A, T-5825-C, T-5930-C. A-6047-G, G-6783-A, G-6963-A, G-7834-A, T-8859-A, T-8864-C, G-9211-A, A-10427-G, G-11560-A, A-11871-G, and T-11978-C. In certain embodiments, the vector lacks nucleotide sequences which encode SFV structural proteins. In certain embodiments, when the vector is propagated in cell culture, titers of at least 10⁷ plaque forming units (pfu) per ml of VLVs are obtained.

In one aspect, the invention provides a method of immunizing a subject against SARS-CoV-2 infection. In certain embodiments, the method comprises administering to the subject a composition comprising at least 10⁷ pfu/ml of the VLVs of the disclosure. In certain embodiments, expression of the SARS-CoV-2 antigen induces an immune response in the subject.

In one aspect, the disclosure provides a method of treating, ameliorating, and/or preventing a disease in a subject. In certain embodiments, the method comprises administering a therapeutically effective amount of the composition of the disclosure to a subject in need of such treatment.

In one aspect, the invention provides a method of vaccinating a subject. In certain embodiments, the method comprises administering to the subject a pharmaceutically acceptable amount of the composition of the disclosure. In certain embodiments, administration of the composition elicits an immune response in the subject.

In one aspect, the invention provides a method of generating a memory T cell immune response to a SARS-CoV-2 antigen or fragment thereof in a subject. In certain embodiments, the method comprises administering the composition of the disclosure to a subject in an amount effective to elicit an immune response in the subject. In certain embodiments, the method comprises administering a second effective amount of the composition of the disclosure at a second, subsequent time period. In certain embodiments, T memory cells directed against the SARS-CoV-2 antigen or fragment thereof are generated in the subject.

In one aspect, the invention provides a method of generating an adaptive B cell immune response to a SARS-CoV-2 antigen or fragment thereof in a subject. In certain embodiments, the method comprises administering the composition of the disclosure to a subject in an amount effective to elicit an immune response in the subject. In certain embodiments, the method comprises administering a second effective amount of the composition of the disclosure at a second, subsequent time period. In certain embodiments, B memory cells directed against the SARS-CoV-2 antigen or fragment thereof are generated in the subject

In one aspect, the invention provides a VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a SARS-CoV-antigen or fragment thereof, operably linked to a 2A DNA encoding a 2A peptide, which is in turn operably linked to a vesicular stomatitis virus (VSV) G DNA encoding a VSV G protein. In certain embodiments, the alphavirus non-structural protein nucleotide sequences comprise at least two of the mutations selected from the group consisting of G-4700-A, A-5424-G, G-5434-A, T-5825-C. T-5930-C, A-6047-G, G-6783-A, G-6963-A, G-7834-A, T-8859-A, T-8864-C, G-9211-A, A-10427-G, G-11560-A, A-11871-G, and T-11978-C. In certain embodiments, the vector lacks nucleotide sequences which encode alphavirus structural proteins. In certain embodiments, when the vector is propagated in cell culture, titers of at least 10⁷ plaque forming units (pfu) per ml of virus like vesicles (VLVs) are obtained.

In one aspect, the invention provides a virus like vesicle (VLV) producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a Glypican 3 (GPC3) protein or fragment thereof, operably linked to a 2A DNA encoding a 2A peptide, which is in turn operably linked to a vesicular stomatitis virus (VSV) G DNA encoding a VSV G protein. In certain embodiments, the SFV non-structural protein nucleotide sequences comprise at least two of the mutations selected from the group consisting of G-4700-A, A-5424-G, G-5434-A, T-5825-C, T-5930-C. A-6047-G, G-6783-A, G-6963-A, G-7834-A, T-8859-A, T-8864-C, G-9211-A, A-10427-G, G-11560-A, A-11871-G, and T-11978-C. In certain embodiments, the vector lacks nucleotide sequences which encode SFV structural proteins. In certain embodiments, when the vector is propagated in cell culture, titers of at least 10⁷ plaque forming units (pfu) per ml of VLVs are obtained.

In one aspect, the invention provides a method of inducing an immune response in a subject against a GPC3 antigen or antigen fragment. In certain embodiments, the method comprises administering to the subject a composition comprising at least 10⁷ pfu/ml of the VLVs of the disclosure. In certain embodiments, expression of the GPC3 antigen induces an immune response in the subject.

In one aspect, the invention provides a method of treating, ameliorating, and/or preventing a disease in a subject. In certain embodiments, the method comprises administering a therapeutically effective amount of the composition of the disclosure to a subject in need thereof.

In one aspect, the invention provides a method of vaccinating a subject. In certain embodiments, the method comprises administering to the subject a pharmaceutically acceptable amount of the composition of the disclosure. In certain embodiments. administration of the composition elicits an immune response in the subject.

In one aspect, the invention provides a method of generating a memory T cell immune response to a GPC3 antigen or fragment thereof in a subject. In certain embodiments, the method comprises administering the composition of the disclosure to a subject in an amount effective to elicit an immune response in the subject. In certain embodiments, the method comprises administering a second effective amount of the composition of the disclosure at a second, subsequent time period. In certain embodiments, T memory cells directed against the GPC3 antigen or fragment thereof are generated in the subject.

In one aspect, the invention provides a method of generating an adaptive B cell immune response to a GPC3 antigen or fragment thereof in a subject. In certain embodiments, the method comprises administering the composition of the disclosure to a subject in an amount effective to elicit an immune response in the subject. In certain embodiments, the method comprises administering a second effective amount of the composition of the disclosure at a second, subsequent time period. In certain embodiments, B memory cells directed against the GPC3 antigen or fragment thereof are generated in the subject.

In one aspect, the invention provides a VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a GPC3 antigen or fragment thereof, operably linked to a 2A DNA encoding a 2A peptide, which is in turn operably linked to a vesicular stomatitis virus (VSV) G DNA encoding a VSV G protein. In certain embodiments, the alphavirus non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C. In certain embodiments, the vector lacks nucleotide sequences which encode alphavirus structural proteins. In certain embodiments, the vector is propagated in cell culture, titers of at least 10⁷ plaque forming units (pfu) per ml of virus like vesicles (VLVs) are obtained.

### BRIEF DESCRIPTION OF THE DRAWINGS

For the purpose of illustrating the disclosure, there are depicted in the drawings certain embodiments of the disclosure. However, the disclosure is not limited to the precise arrangements and instrumentalities of the embodiments depicted in the drawings.

FIGS. 1A-1C illustrate a virus-like vesicle platform for COVID-19 vaccine expressing RBD or full length of SARS-CoV-2 spike protein.

FIGS. 2A-2C illustrate anti-spike protein antibody responses in the serum of C57BL/6 mice immunized with PBS, VLV-RBD or VLV-Full.

FIG. 3 illustrates the effects of purified spike protein on CD4+ T cell proliferation. Splenocytes isolated from the C57BL/6 mice immunized with PBS. VLV-RBD or VLV-Full on day 0.

FIGS. 4A-4C illustrate the effects of injection methods on anti-spike protein immune responses.

FIGS. 5A-5C illustrate a comparison of antibody responses in C57BL/6 mice immunized with VLV-Full or Pfizer mRNA vaccine.

FIG. 6 illustrates a neutralization assay.

FIG. 7 is a diagram of two VLV constructs comprising the SARS-CoV-2 full-length spike protein or the spike protein receptor binding domain (RBD) for use in vaccination.

FIGS. 8A-8C illustrate a virus-like vesicle platform for HCC vaccine expressing Glypican-3 (GPC3). FIG. 8A: Design of replicating VLV for expression of GPC3. The plasmid construction contains T2A self-cleaving peptide that allow it to express both GPC3 and VSV-G protein. FIG. 8B: Evaluation of GPC3 expression in BHK-21 cells after infection with VLV-GPC3 in western blot. VLV-ctrl infected BHK-21 cells were collected and used as negative control. FIG. 8C: Validation of VSV-G expression in BHK-21 cells after infection with VLV-GPC3 (MOI=1) by immunofluorescence at 24 h post infection.

FIGS. 9A-9D illustrate anti-GPC3 antibody responses in the serum of C57BL/6 mice immunized with PBS, VLV-ctrl or VLV-GPC3. FIG. 9A: Experimental procedure. FIG. 9B: Antibody levels, expressed as OD value, in the serum of C57BL/6 mice (n=8) on day 26 after the immunization with PBS, VLV-ctrl (1×10⁸ PFU/mice) or VLV-GPC3 (1×10⁸ PFU/mice). FIG. 9C: Antibody series dilution in the serum of C57BL/6 mice (n=8) on day 26 after the immunization with VLV-GPC3 (1×10⁸ PFU/mice). FIG. 9D: The ratio of IgG2a/IgG1 in the serum of C57BL/6 mice (n=8) on day 26 after the immunization with VLV-GPC3 (1×10⁸ PFU/mice). Bar represents mean±standard deviation (SD). *, P<0.05: **, P<0.01: ***, P<0.005: ****, P<0.001.

FIG. 10 illustrates an ELISPOT analysis of IFN-γ secretion in the immunized mouse splenocytes incubated with the purified GPC3. Splenocytes isolated from the immunized C57BL/6 mice were incubated with purified GPC3 (5 μg/ml) for detecting IFN-γ production at 96 h. The splenocytes were collected 70 days after VLV-GPC3 immunization. Representative wells are shown after plate development. Each wells contained 3×10⁵ cells. Bar represents mean±standard deviation (SD). ****, P<0.001.

FIGS. 11A-11D illustrate preventative effects against Hepa1-6 tumor cells in mice immunized with VLV-GPC3. FIG. 11A: Experimental procedure. FIG. 11B: Tumor sizes were measured 3 to 4 times a week and calculated using the equation V=length×width²/2. FIGS. 11C-11D: Flow cytometry was performed on day 28 to analyze CD4+ T and CD8+ T cell percentage in blood. Bar represents mean±standard deviation (SD). *. P<0.05: **P<0.01: ***P<0.005: ****, P<0.001.

FIGS. 12A-12D illustrate therapeutic effects against Hepa1-6 tumor cells in mice. FIG. 12A: Experimental procedure. FIG. 12B: Tumor volumes were measured 3-4 times a week using a caliper and calculated using the equation V=length×width²/2. FIGS. 12C-12D: Flow cytometry was performed on day 28 to analyze CD4+ T and CD8+ T cell percentage in blood. Bar represents mean±standard deviation (SD). *, P<0.05; **, P<0.01: ***P<0.005: ****, P<0.001.

FIG. 13 is a diagram of a GPC3 VLV construct and its use in preventive and therapeutic treatment settings.

### DETAILED DESCRIPTION OF DISCLOSURE

The present disclosure relates in one aspect to compositions and/or methods for promoting therapeutic immunization in the treatment of SARS-CoV-2 infections and/or diseases associated with SARS-CoV-2 infection, including COVID-19. In various embodiments described herein, the methods of the disclosure include generating virus-like vesicles (VLVs) from VLV producing vectors that expresses SARS-CoV-2 structural proteins, wherein the vector produces high titers of virus like vesicles (VLVs). Additionally, the present disclosure includes methods of treating, ameliorating, preventing, and/or immunizing a subject against SARS-CoV-2 infection, and methods of generating a memory T and B cell immune responses against SARS-CoV-2 infection in a subject, comprising administering to the subject effective amounts of the VLVs generated by the vectors of the disclosure.

The present invention relates in one aspect to the discovery of compositions and methods for the treatment, amelioration, and/or prevention of cancers associated with expression or and/overexpression of the gene GPC3, especially hepatocellular carcinoma (HCC). In various embodiments described herein, the methods of the disclosure include generating virus-like vesicles (VLVs) from VLV producing vectors that express and/or comprise nucleic acids encoding the GPC3 protein, wherein the vector produces high titers of virus like vesicles (VLVs). Additionally, the present disclosure includes methods of generating a memory T and B cell immune responses against GPC3 in a subject comprising administering effective amounts of the VLVs generated by the vectors of the disclosure.

### Definitions

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

It is also to be understood that the terminology used herein is for the purpose of describing particular embodiments only, and is not intended to be limiting.

The terms “2A” or “2A peptide” or “2A-like peptide” is a self-processing viral peptide. The 2A peptide can separate different protein coding sequences in a single ORF transcription unit (Ryan et al., 1991, J Gen Virol 72:2727-2732). Although termed a “self-cleaving” peptide or protease site, the mechanism by which the 2A sequence generates two proteins from one transcript occurs by ribosome skipping where a normal peptide bond is impaired at 2A, resulting in two discontinuous protein fragments from one translation event. Linking with 2A peptide sequences results in cellular expression of multiple, discrete proteins (in essentially equimolar quantities) derived from a single ORF (de Felipe et al., 2006, Trends Biotechnol 24:68-75).

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%. more preferably ±5%, even more preferably ±1%, and still more preferably ±0.1% from the specified value, as such variations are appropriate to perform the disclosed methods.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process. The term “activated T cells” indicates T cells that are currently undergoing cell division, cytokine production, performance of regulatory or cytolytic effector functions, and/or has recently undergone the process of “activation.”

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete. “Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymeres, bacterial ghosts, bacterial polysaccharides, attenuated bacterias, virus like particles, attenuated viruses and ISCOMS.

As defined herein, an “Alphavirus” is a member of the Group IV Togaviridae family of viruses. Alphaviruses include, but are not limited to Aura virus, Babanki virus, Barmah Forest virus, Bebaru virus, Cabassou virus, Chikungunya virus, Eastern equine encephalitis virus. Everglades virus, Fort Morgan virus, Getah virus. Highlands virus, Kyzylagach virus, Mayaro virus. Me Tri virus. Middelburg virus. Mosso das Pedras virus, Mucambo virus. Ndumu virus, O'nyong nyong virus, Pixuna virus, Rio Negro virus. Ross River virus, Sagiama virus, Salmon pancreas disease virus, Semliki Forest virus, Sindbis virus, Southern elephant seal virus. Tonate virus, Trocara virus, Una virus, Venezuelan equine encephalitis virus. Western equine encephalitis virus and Whataroa virus.

As herein defined, an “alphavirus non-structural protein” can be selected from the group consisting of nsp1, nsp2, nsp3 and nsp4.

As defined herein, an “alphavirus structural protein” can be selected from the group consisting of an alphavirus capsid protein and at least one spike protein.

The term “ameliorating” or “treating” means that the clinical signs and/or the symptoms associated with a disease are lessened as a result of the actions performed. The signs or symptoms to be monitored will be well known to the skilled clinician.

The term “antibody” or “Ab” as used herein, refers to a protein, or polypeptide sequence derived from an immunoglobulin molecule which specifically binds to a specific epitope on an antigen. Antibodies can be intact immunoglobulins derived from natural sources or from recombinant sources and can be immunoreactive portions of intact immunoglobulins. The antibodies useful in the present disclosure may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual. Cold Spring Harbor Laboratory Press, NY: Harlow et al., 1989. Antibodies: A Laboratory Manual, Cold Spring Harbor, New York: Houston et al., 1988, Proc. Natl. Acad. Sci. USA 85:5879-5883: Bird et al., 1988, Science 242:423-426). An antibody may be derived from natural sources or from recombinant sources. Antibodies are typically tetramers of immunoglobulin molecules.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, by “combination therapy” is meant that a first agent is administered in conjunction with another agent. “In conjunction with” or “in combination with” refers to administration of one treatment modality in addition to another treatment modality. As such, “in conjunction with” or “in combination with” refers to administration of one treatment modality before, during, or after delivery of the other treatment modality to the individual. Such combinations are considered to be part of a single treatment regimen or regime.

As used herein, the term “concurrent administration” means that the administration of the first therapy and that of a second therapy in a combination therapy overlap with each other.

A “constitutive” promoter is a nucleotide sequence which, when operably linked with a polynucleotide which encodes or specifies a gene product, causes the gene product to be produced in a cell under most or all physiological conditions of the cell.

As used herein, the terms “control” or “reference” are used interchangeably, and refer to a value that is used as a standard of comparison.

The term “coronavirus” as used herein refers to a member of Coronaviridae, a family of enveloped, positive-sense single-strand RNA viruses. Coronaviruses can cause disease in birds and mammals. One of the most well-studied coronaviruses is the murine coronavirus MHV, which causes epidemic infections in laboratory animals. In humans, coronaviruses typically cause respiratory infections that range in severity from the common cold to more lethal diseases such as SARS, Middle East Respiratory Syndrome (MERS), and COVID-19. In certain embodiments, the virus comprises a Coronavirus. In certain embodiments, the Coronavirus comprises an Alphacoronavirus, a Betacoronavirus, a Gammacoronavirus, and/or a Deltacoronavirus. In certain embodiments, the Coronavirus is an Alphacoronavirus, such as but not limited to Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, and/or Scotophilus bat coronavirus 512. In certain embodiments, the Coronavirus is a Betacoronavirus, such as but not limited to Betacoronavirus 1 (Bovine Coronavirus. Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), and/or Tylonycteris bat coronavirus HKU4. In certain embodiments, the Coronavirus is a Severe acute respiratory syndrome-related coronavirus (SARS-CoV, SARS-CoV-2). In certain embodiments, the Coronavirus is a Severe acute respiratory syndrome-related coronavirus, SARS-CoV-2. In certain embodiments, the Coronavirus is a Gammacoronavirus, such as but not limited to Avian coronavirus and/or Beluga whale coronavirus SW1. In certain embodiments, the Coronavirus is a Deltacoronavirus, such as but not limited to Bulbul coronavirus HKU11 and/or Porcine coronavirus HKU15. In certain embodiments, the Coronavirus comprises at least one of MERS-CoV, SARS-CoV, and/or SARS-CoV 2. In certain embodiments, the SARS-CoV-2 comprises at least one variant selected from B.1.1.7 (Alpha), B.1.351 (Beta), P.1 (Gamma), B.1.617.2 (Delta), B.1.429/B.1.427 (Epsilon), B.1.617.1 (Kappa). B.1.525 (Eta), B.1.526 (Iota), P.3 (Theta). P.2 (Zeta), and B.1.1.529 (Omicron). In certain embodiments, the SARS-CoV-2 comprises at least one variant selected from A.1-A.6, B.3-B.7, B.9, B.10, B.13-B. 16, B.2, B.1 lineage (including, but not limited to, B.1. B.1.1, B.1.1.7, B.1.1.7 with E484K, B.1.2, B.1.5-B.1.72, B.1.9, B.1.13, B.1.22, B.1.26, B.1.37, B.1.3-B.1.66, B.1.177, B.1.243, B.1.313, B.1.351, B.1.427, B.1.429, B.1.525, B.1.526, B.1.526.1, B.1.526.2, B.1.617, B.1.617.1, B.1.617.2, B.1.617.3, B.1.619, B.1.620, and B.1.621), P.1, P.2, P.3, and R.1.

The term “coronavirus disease 2019” or COVID-19, as used herein, refers to the disease caused initially by infection of a subject with the novel coronavirus also known as severe acute respiratory syndrome coronavirus 2 (SARS-CoV-2). COVID-19, while caused initially by the infection with the SARS-CoV-2, is characterized in that it triggers a severe immune response in a subpopulation of individuals. The immune response to the SARS-CoV-2 virus and to the cells infected therefrom, in combination with the damage to the cells of the lung caused by the SARS-CoV-2 virus itself, can lead to acute disease, including acute respiratory distress syndrome in a subset of patients. COVID-19 can thereby require intubation, mechanical ventilation, and/or the use of a heart and lung bypass machine in a further subset of patients.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “effective amount” or “therapeutically effective amount” means the amount of the virus like vesicle generated from vector of the disclosure which is required to prevent the particular disease condition, or which reduces the severity of and/or ameliorates the disease condition or at least one symptom thereof or condition associated therewith.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

The term “evolved” or “evolve” as used herein refers to the change (i.e. the evolution) in the inherited characteristics of biological populations over successive generations.

Evolutionary processes give rise to diversity at every level of biological organization, including species, individual organisms and molecules such as DNA and proteins (Hall and Hallgrimsson, eds. 2008, Strickberger's Evolution (4th ed.). Jones & Bartlett). In the context of the present disclosure, the “evolved” hybrid-virus accumulated beneficiary mutations and produced VLVs with 1000 times higher titers after 50 passages in culture than the original hybrid virus.

A “fusion protein” as used herein refers to a protein wherein the protein comprises two or more proteins linked together by peptide bonds or other chemical bonds. The proteins can be linked together directly by a peptide or other chemical bond, or with one or more amino acids between the two or more proteins, referred to herein as a spacer.

As used herein. “greater” refers to expression levels which are at least 10% or more. for example, 20%, 30%, 40%, or 50%. 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control.

“Humoral immunity” or “humoral immune response” both refer to B-cell mediated immunity and are mediated by highly specific antibodies, produced and secreted by B-lymphocytes (B-cells).

“Heterologous antigens” used herein to refer to an antigen that is not endogenous to the organism comprising or expressing an antigen. As an example, a virus vaccine vector comprising or expressing a viral or tumor antigen comprises a heterologous antigen.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

“Incorporated into” or “encapsulated in” refers to an antigenic peptide that is within a delivery vehicle, such as microparticles, bacterial ghosts, attenuated bacteria, virus like particles, attenuated viruses, ISCOMs, liposomes and preferably virosomes.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. An “isolated cell” or “isolated population of cells” is a cell or population of cells that is not present in its natural environment.

A “mutation” as used therein is a change in a DNA sequence resulting in an alteration from its natural state. The mutation can comprise deletion and/or insertion and/or duplication and/or substitution of at least one deoxyribonucleic acid base such as a purine (adenine and/or thymine) and/or a pyrimidine (guanine and/or cytosine)

Mutations may or may not produce discernible changes in the observable characteristics (phenotype) of an organism (subject).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide.” “polypeptide.” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “pharmaceutical composition” refers to a mixture of at least one compound useful within the disclosure with other chemical components, such as carriers. stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

“Prevention” refers to the use of a pharmaceutical compositions for the vaccination against a disorder.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “RNA” as used herein is defined as ribonucleic acid.

The term “specifically binds”, “selectively binds” or “binding specificity” refers to the ability of the humanized antibodies or binding compounds of the disclosure to bind to a target epitope present on VSV with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

A “subject” or “patient,” as used therein, may be a human or non-human mammal. Non-human mammals include, for example, livestock and pets, such as ovine, bovine, porcine, canine, feline and murine mammals. In certain embodiments, the subject is human.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on HeLa cells using the soft agar method (see, Graham & Van Der eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

As used herein, the term “transfection” includes any means known to the skilled artisan where nucleic sequences are delivered into a cell. Methods of transfecting nucleic acids into cells are described, for example, in Sambrook et al. “Molecular Cloning”, A laboratory manual, Cold Spring Harbor Laboratory Press, Volumes 1-3, 2001 (ISBN-0879695773). Typical transfection methods include electroporation and use of lipids or calcium phosphate.

“Transduce”, “transducing”, and “transduction” is used herein to refer to a process of introducing an isolated nucleic acid into the interior of a cell or organism, particularly by the use of viral vectors.

The term “treatment” as used within the context of the present disclosure is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. Treatment may include curative and/or alleviative effects. References herein to “therapeutic” and “prophylactic” treatments are to be considered in their broadest context. The term “therapeutic” does not necessarily imply that a subject is treated until total recovery. Thus, for example, the term treatment includes the administration of an agent following the onset of a disease or disorder thereby removing all signs of the disease or disorder. As another example, administration of the agent after clinical manifestation of the disease to combat the symptoms of the disease comprises “treatment” of the disease.

“Vaccination” refers to the process of inoculating subject with an antigen to elicit an immune response in the subject, that helps to prevent or treat the disease or disorder the antigen is connected with. The term “immunization” is used interchangeably herein with vaccination.

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. The polypeptides generally will have significant amino acid identity relative to each other. A polymorphic variant is a variation in the polynucleotide sequence of a particular gene between individuals of a given species. Polymorphic variants may encompass “single nucleotide polymorphisms” (SNPs) in which the polynucleotide sequence varies by one base. The presence of SNPs may be indicative of, for example, a certain population, a disease state, or a propensity for a disease state.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

The term “virus like vesicle” or “VLV” as used herein refers to a form of self-amplifying viral replicons that are non-pathogenic relative to viral vectors while also retaining immunogenicity. VLVs are capable of inducing robust T cell and antibody responses while retaining a desirable safety profile for clinical use. The RNA-dependent RNA polymerase from Semliki Forest virus and glycoprotein from vesicular stomatitis virus (VSV-G) expressed from the same vector are essential components of the VLV platform that enables rapid and efficient production of VLVs in eukaryotic cells and expression of the desired antigens in the subject.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### SARS-CoV-2

The disclosure is based in part on the discovery of the high titer hybrid-virus VLV producing vectors comprising one or more SARS-CoV-2 antigens or antigen fragments. The VLV producing vector, which has been previously described in U.S. Pat. No. 9,987,353, which is hereby incorporated in its entirety by reference, comprises a DNA sequence comprising a first promoter sequence operably linked to a DNA sequence encoding Semliki Forest virus (SFV) non-structural protein nucleotide sequences comprising at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C. These sequences are operably linked to DNA specifying a second promoter sequence corresponding to the SFV subgenomic RNA promoter. This sequence is in turn operably linked to DNA encoding a vesicular stomatitis virus (VSV) G protein. The vector lacks functional nucleotide sequences which encode SFV structural proteins. When the vector is propagated in cell culture, high titers of virus like vesicles (VLVs) are obtained, for example, titers of at least 10⁷ plaque forming units (pfu) per ml are obtained.

In some embodiments, the vector of this disclosure comprises a DNA sequence comprising a first promoter sequence operably linked to a DNA sequence encoding alphavirus non-structural protein nucleotide sequences comprising at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C. These sequences are operably linked to DNA specifying a second promoter sequence corresponding to the alphavirus subgenomic RNA promoter. This sequence is in turn operably linked to DNA encoding a vesicular stomatitis virus (VSV) G protein. The vector lacks functional nucleotide sequences which encode SFV structural proteins. When the vector is propagated in cell culture, high titers of virus like vesicles (VLVs) are obtained, for example, titers of at least 10⁷ plaque forming units (pfu) per ml are obtained. These VLVs comprise both protein antigens and nucleic acids encoding the antigens of interest (e.g. SARS-CoV-2 antigens or antigen fragments),

Methods of making the VLV producing vector of the disclosure can be known in the art (such as, but not limited to, in U.S. Pat. No. 9,987,353) and/or are described in detail in the Experimental Examples section herein.

In one aspect, the VSV encoding the VSV G protein can be from any VSV serotype known in the art. Non-limiting examples of VSV serotypes include the Indiana (IND-VSV) serotype and New Jersey (NJ-VSV) serotype.

In one aspect, although the cytomegalovirus immediate early promoter is exemplified elsewhere herein, the disclosure should not be construed to be limited to this promoter sequence. Promoter sequences that are useful in the disclosure include any promoter that induces high levels of gene expression. Such promoters may include, but are not limited to those disclosed elsewhere herein.

In another aspect of the disclosure, the hybrid-virus vector may achieve titers of at least 5×10⁷ pfu/ml, at least 1×10⁸ pfu/ml or more when the hybrid-virus vector is propagated in cell culture.

In a further aspect of the composition of the disclosure, DNA encoding a SARS-CoV-2 protein antigen is inserted between the subgenomic SFV promoter and DNA encoding the VSV G protein wherein the DNA encoding the heterologous protein is operably linked to DNA encoding a T2A peptide from Thosea asigna virus (Szymczak et al., 2004, Nature Biotechnology 22:589-594) which is in turn operably linked to DNA encoding the VSV G protein. In this way, expression of the SARS-CoV-2 protein antigen in the resulting VLVs of the disclosure is effectively tied to expression of the VSV G protein, the latter being essential for replication of the vector. Thus, expression of the SARS-CoV-2 protein is stabilized and the continued presence of the gene expressing this protein in the hybrid vector is assured.

In some embodiments, the 2A peptide is selected from the group consisting of equine rhinitis A virus (E2A), foot-and-mouth disease virus (F2A), porcine teschovirus-1 (P2A), Thosea asigna virus (T2A) and any 2A peptide or fragment thereof known in the art. In further embodiments, the 2A peptide is a T2A peptide or any T2A fragment thereof known in the art.

In certain embodiments, the SARS-CoV-2 gene can be under the control of an RNA virus promoter sequence that may not necessarily be the SFV subgenomic promoter sequence. Such modifications and variations of the RNA promoter sequence driving expression of the heterologous promoter sequences will become apparent to those skilled in the art as they practice the disclosure. The vector may also include conventional control elements which are operably linked to the SARS-CoV-2 gene in a manner which permits its transcription, translation and/or expression in a cell infected with the VLV producing vector produced by the disclosure.

In this way, expression of the SARS-CoV-2 protein/antigen or fragment thereof is effectively tied to expression of the VSV G protein, the latter being essential for replication of the vector. Thus, expression of the SARS-CoV-2 antigen or fragment thereof is stabilized and the continued presence of the gene expressing this protein in the hybrid vector is assured. Such a high titer hybrid-virus vector is referred to herein as a “VLV producing SARS-CoV-2 vector.”

In certain embodiments, the DNA encoding the SARS-CoV-2 antigen or fragment thereof is under the control of a constitutive promoter. In other embodiments, the required component(s) may be under the control of an inducible promoter. Examples of suitable inducible and constitutive promoters are provided elsewhere herein, and are well known in the art.

The vector may also include conventional control elements which are operably linked to the heterologous gene in a manner which permits its transcription, translation and/or expression in a cell infected with the hybrid-virus vector produced by the disclosure.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the disclosure. “Operably linked” should be construed to include RNA expression and control sequences in addition to DNA expression and control sequences.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements may function either cooperatively or independently to activate transcription.

In certain embodiments, a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used. including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. The disclosure further includes the use of tissue-specific promoter that drive expression of a given heterologous gene in one or more specific types of cells (e.g., desmin promoter, myoglobin promoter, muscle creatine kinase promoter, mammalian troponin 1 promoter, and skeletal alpha-action promoter). Furthermore, any artificial synthetic promoters known in the art can be used in this disclosure as these promoters can provide optimal efficiency and stability for the heterologous gene. Additionally, enhancer sequences regulates expression of the gene contained within a vector. Typically, enhancers are bound with protein factors to enhance the transcription of a gene. Enhancers may be located upstream or downstream of the gene it regulates. Enhancers may also be tissue-specific to enhance transcription in a specific cell or tissue type.

In order to assess the expression of the DNA encoding the SARS-CoV-2 antigen, the expression vector to be introduced into a cell can also contain either a selectable marker gene or a reporter gene or both to facilitate identification and selection of expressing cells from the population of cells sought to be infected through the hybrid-virus vectors. In other aspects, the selectable marker may be carried on a separate piece of DNA and used in a co-infection/transfection procedure. Both selectable markers and reporter genes may be flanked with appropriate regulatory sequences to enable expression in the host cells. Useful selectable markers include, for example, antibiotic-resistance genes, such as the neomycin resistant gene and the like.

Reporter genes are used for identifying potentially infected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

The SARS-CoV-2 antigens useful in the high titer hybrid-SARS-CoV-2 vector of the disclosure include SARS-CoV-2 spike protein, a fragment of the SARS-CoV-2 spike protein, the SARS-CoV-2 spike protein receptor binding domain (RBD), SARS-CoV-2 nucleocapsid protein, a fragment of the SARS-CoV-2 nucleocapsid protein, and any combination thereof. In further aspects, the SARS-CoV-2 antigen could be derived from any known in the art variant or serotype, or genotype as described elsewhere herein. In yet further aspects, the SARS-CoV-2 antigen could encompass combinations of SARS-CoV-2 antigens (i.e. 2, 3 or more) or a fragment thereof. In some aspects these SARS-CoV-2 antigens or SARS-CoV-2 antigen fragments can be assembled into a fusion protein using technology available in the art.

In another aspect of the disclosure, the VLV producing SARS-CoV-2 vector may produce VLVs expressing SARS-CoV-2 at titers of at least 5×10⁷ pfu/ml, at least 1×10⁸ pfu/ml or more when the VLVs are produced in cell culture.

### HCC

The protein product of the Glypican-3 or GPC3 gene is a membrane-bound heparan sulfate proteoglycan protein. GPC3 is a member of a family of six GPC proteins, each consisting of a 60-70 kDa protein which is associated to the cell membrane via a glycosylphosphatidylinositol (GPI) anchor. GPC3 participates in a number of cellular process including cell growth, differentiation, and migration. GPC3 has been found to be strongly expressed or overexpressed in a variety of solid cancers, including hepatocellular carcinoma (HCC), ovarian clear cell carcinoma, melanoma, squamous cell carcinoma of the lung, hepatoblastoma, nephroblastoma (Wilms tumor), yolk sac tumor, and some pediatric cancers among others. In hepatocellular carcinoma (HCC), strong GPC3 expression on tumor cells is accompanied with little or no expression on normal or non-cancerous (e.g. cirrhotic) liver tissues. The selectivity of GPC3 expression on HCC cells makes the molecule an attractive target for immunotherapies seeking to treating HCC.

The present disclosure is based in part on the discovery of high titer hybrid-virus VLV producing vectors comprising Glypican-3 (GPC3) antigens or antigen fragments that can be used to treat, ameliorate, and/or prevent cancers associated with expression and/or overexpression of GPC3 protein, including but not limited to HCC. The VLV producing vector, which has been previously described in U.S. Pat. No. 9,987,353, which is hereby incorporated by reference in its entirety, comprises a DNA sequence comprising a first promoter sequence operably linked to a DNA sequence encoding Semliki Forest virus (SFV) non-structural protein nucleotide sequences comprising at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C. These sequences are operably linked to DNA specifying a second promoter sequence corresponding to the SFV subgenomic RNA promoter. This sequence is in turn operably linked to DNA encoding a vesicular stomatitis virus (VSV) G protein. The vector lacks functional nucleotide sequences which encode SFV structural proteins. When the vector is propagated in cell culture, high titers of virus like vesicles (VLVs) are obtained, for example, titers of at least 10⁷ plaque forming units (pfu) per ml are obtained.

In some embodiments, the vector of this disclosure comprises a DNA sequence comprising a first promoter sequence operably linked to a DNA sequence encoding alphavirus non-structural protein nucleotide sequences comprising at least two of the mutations selected from the group consisting of G-4700-A, A-5424-G. G-5434-A. T-5825-C, T-5930-C, A-6047-G, G-6783-A, G-6963-A, G-7834-A, T-8859-A, T-8864-C, G-9211-A, A-10427-G, G-11560-A. A-11871-G, and T-11978-C. These sequences are operably linked to DNA specifying a second promoter sequence corresponding to the alphavirus subgenomic RNA promoter. This sequence is in turn operably linked to DNA encoding a vesicular stomatitis virus (VSV) G protein. The vector lacks functional nucleotide sequences which encode SFV structural proteins. When the vector is propagated in cell culture, high titers of virus like vesicles (VLVs) are obtained, for example, titers of at least 10⁷ plaque forming units (pfu) per ml are obtained. These VLVs comprise both protein antigens and nucleic acids encoding the antigens of interest (e.g. GPC3 antigens or antigen fragments),

Methods of making the VLV producing vector of the disclosure are described in detail in the prior art (such as but not limited to U.S. Pat. No. 9,987,353) and/or in the Experimental Examples Section herein.

In one aspect, the VSV encoding the VSV G protein can be from any VSV serotype known in the art. Non-limiting examples of VSV serotypes include the Indiana (IND-VSV) serotype and New Jersey (NJ-VSV) serotype.

In one aspect, although the cytomegalovirus immediate early promoter is exemplified elsewhere herein, the disclosure should not be construed to be limited to this promoter sequence. Promoter sequences that are useful in the disclosure include any promoter that induces high levels of gene expression. Such promoters may include, but are not limited to those disclosed elsewhere herein.

In another aspect of the disclosure, the hybrid-virus vector may achieve titers of at least 5×10⁷ pfu/ml, at least 1×10⁸ pfu/ml or more when the hybrid-virus vector is propagated in cell culture.

In a further aspect of the composition of the disclosure, DNA encoding a GPC3 protein antigen is inserted between the subgenomic SFV promoter and DNA encoding the VSV G protein wherein the DNA encoding the heterologous protein is operably linked to DNA encoding a T2A peptide from Thosea asigna virus (Szymczak et al., 2004, Nature Biotechnology 22:589-594) which is in turn operably linked to DNA encoding the VSV G protein. In this way, expression of the GPC3 protein antigen in the resulting VLVs of the disclosure is effectively tied to expression of the VSV G protein, the latter being essential for replication of the vector. Thus, expression of the GPC3 protein is stabilized and the continued presence of the gene expressing this protein in the hybrid vector is assured.

In some embodiments, the 2A peptide is selected from the group consisting of equine rhinitis A virus (E2A), foot-and-mouth disease virus (F2A), porcine teschovirus-1 (P2A), Thosea asigna virus (T2A) and any 2A peptide or fragment thereof known in the art. In further embodiments, the 2A peptide is a T2A peptide or any T2A fragment thereof known in the art.

In certain embodiments, the GPC3 gene can be under the control of an RNA virus promoter sequence that may not necessarily be the SFV subgenomic promoter sequence. Such modifications and variations of the RNA promoter sequence driving expression of the heterologous promoter sequences will become apparent to those skilled in the art as they practice the disclosure. The vector may also include conventional control elements which are operably linked to the GPC3 gene in a manner which permits its transcription, translation and/or expression in a cell infected with the VLV producing vector produced by the disclosure.

In this way, expression of the GPC3 protein/antigen or fragment thereof is effectively tied to expression of the VSV G protein, the latter being essential for replication of the vector. Thus, expression of the GPC3 antigen or fragment thereof is stabilized and the continued presence of the gene expressing this protein in the hybrid vector is assured. Such a high titer hybrid-virus vector is referred to herein as a “VLV producing GPC3 vector.”

In certain embodiments, the DNA encoding the GPC3 antigen or fragment thereof is under the control of a constitutive promoter. In other embodiments, the required component(s) may be under the control of an inducible promoter. Examples of suitable inducible and constitutive promoters are provided elsewhere herein, and are well known in the art.

The vector may also include conventional control elements which are operably linked to the heterologous gene in a manner which permits its transcription, translation and/or expression in a cell infected with the hybrid-virus vector produced by the disclosure.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the disclosure “Operably linked” should be construed to include RNA expression and control sequences in addition to DNA expression and control sequences.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, individual elements may function either cooperatively or independently to activate transcription.

In certain embodiments, a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter. The disclosure further includes the use of tissue-specific promoter that drive expression of a given heterologous gene in one or more specific types of cells (e.g., desmin promoter, myoglobin promoter, muscle creatine kinase promoter, mammalian troponin 1 promoter, and skeletal alpha-action promoter). Furthermore, any artificial synthetic promoters known in the art can be used in this disclosure as these promoters can provide optimal efficiency and stability for the heterologous gene. Additionally, enhancer sequences regulates expression of the gene contained within a vector. Typically, enhancers are bound with protein factors to enhance the transcription of a gene. Enhancers may be located upstream or downstream of the gene it regulates. Enhancers may also be tissue-specific to enhance transcription in a specific cell or tissue type.

In order to assess the expression of the DNA encoding the GPC3 antigen, the expression vector to be introduced into a cell can also contain either a selectable marker gene or a reporter gene or both to facilitate identification and selection of expressing cells from the population of cells sought to be infected through the hybrid-virus vectors. In other aspects, the selectable marker may be carried on a separate piece of DNA and used in a co-infection/transfection procedure. Both selectable markers and reporter genes may be flanked with appropriate regulatory sequences to enable expression in the host cells. Useful selectable markers include, for example, antibiotic-resistance genes, such as the neomycin resistant gene and the like.

Reporter genes are used for identifying potentially infected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

The GPC3 antigens useful in the high titer hybrid-GPC3 vector of the disclosure include GPC3 protein, a fragment of the GPC3 protein. In further aspects, the GPC3 antigen could be derived from any known in the art variant or genotype as described elsewhere herein.

In another aspect of the disclosure, the VLV producing GPC3 vector may produce VLVs expressing GPC3 at titers of at least 5×10⁷ pfu/ml, at least 1×10⁸ pfu/ml or more when the VLVs are produced in cell culture.

### Methods

In certain aspects, the present disclosure includes a method of immunizing and/or vaccinating a subject against infection with SARS-CoV-2. The method comprises administering to the subject a composition comprising virus like vesicles (VLVs) produced by the VLV producing vectors of the disclosure, wherein VLV producing vector comprises DNA encoding a SARS-CoV-2 gene or fragment thereof. Expression of the heterogeneous gene induces an immune response to the SARS-CoV-2 protein or fragment thereof encoded thereby in the subject. In certain embodiments, the SARS-CoV-2 protein or fragment thereof is the full-length spike protein (s protein). In certain embodiments, the SARS-CoV-2 protein or fragment thereof is the receptor binding domain (RBD) of the spike protein. In certain embodiments, the SARS-CoV-2 protein is the nucleocapsid protein. It is also contemplated that in certain embodiments, the VLVs of the disclosure may comprise any combination of SARS-CoV-2 proteins or fragments thereof disclosed herein. For example, VLVs produced by the VLV producing vectors of the disclosure may comprise both SARS-CoV-2 spike and nucleocapsid proteins.

In certain embodiments, a method for reducing an amount of SARS-CoV-2 mRNA, DNA, protein and/or an amount of SARS-CoV-2 antigen in a mammal infected with a SARS-CoV-2 virus is provided. The method comprises administering to a mammal in need thereof a therapeutically effective amount of a composition comprising the virus like vesicles (VLVs) as described herein so as to reduce the SARS-CoV-2 virus infection and the SARS-CoV-2antigen, compared to the amount of SARS-CoV-2 mRNA, protein and an amount of SARS-CoV-2 antigen in the mammal before treatment.

In one aspect, the disclosure includes a method of generating a memory T cell immune response to the SARS-CoV-2 protein or fragment thereof in the subject. In certain embodiments, the method comprises administering a composition comprising VLVs produced by the VLV-producing vector of the disclosure to a subject in an amount effective to elicit an immune response in the subject. In certain embodiments, the method comprises administering a second effective amount of the composition of VLVs produced by the VLV producing vector of the disclosure at a second, subsequent time period, wherein T memory cells directed against the SARS-CoV-2 antigen or fragment thereof are generated in the subject In this way, the production of memory T cells specific for the SARS-CoV-2 protein or fragment thereof are sufficient to help clear an existing SARS-CoV-2 infection in the subject or to provide immunity against a future infection with SARS-CoV-2 virus. In certain embodiments, the SARS-CoV-2 antigen or fragment thereof is full-length spike protein. In certain embodiments, the SARS-CoV-2 protein is the nucleocapsid protein. It is also contemplated that in certain embodiments, the VLVs of the disclosure may comprise any combination of SARS-CoV-2 proteins or fragments thereof disclosed herein. For example, VLVs produced by the VLV producing vectors of the disclosure may comprise both SARS-CoV-2 spike and nucleocapsid proteins.

In another aspect, the disclosure includes a method of generating an adaptive B cell immune response the SARS-CoV-2 protein or fragment thereof in the subject. In certain embodiments, the method comprises administering a composition comprising VLVs produced by the VLV producing vector of the disclosure to a subject in an amount effective to elicit an immune response in the subject. In certain embodiments, the method comprises administering a second effective amount of the composition of claim 5 at a second, subsequent time period, wherein B memory cells directed against the SARS-CoV-2 antigen or fragment thereof are generated in the subject. In this way, the production of memory B cells specific for the SARS-CoV-2 protein or fragment thereof produce antibodies which are sufficient to help clear an existing SARS-CoV-2 infection in the subject or to provide immunity against a future infection with SARS-CoV-2 virus. In certain embodiments, the SARS-CoV-2 antigen or fragment thereof is full-length spike protein. In certain embodiments, the SARS-CoV-2 protein is the nucleocapsid protein. It is also contemplated that in certain embodiments, the VLVs of the disclosure may comprise any combination of SARS-CoV-2 proteins or fragments thereof disclosed herein. For example, VLVs produced by the VLV producing vectors of the disclosure may comprise both SARS-CoV-2 spike and nucleocapsid proteins.

In another aspect, the disclosure includes a method of treating and/or preventing a disease in a subject in need thereof wherein the method comprises administering to the subject a composition comprising virus like vesicles (VLVs) produced by the VLV producing vector of the disclosure, wherein the VLV producing vector comprises DNA encoding a SARS-CoV-2 gene or fragment thereof and wherein expression of the SARS-CoV-2 gene or fragment thereof induces an immune responses against the SARS-CoV-2 gene or fragment thereof that is of benefit to the subject. In certain embodiments, the disease is related to a SARS-CoV-2 infection. In certain preferred embodiments, the disease is COVID-19.

Additionally, in another aspect, the disclosure includes a method of diminishing the risk that a subject will develop a disease associated with a SARS-CoV-2 infection. The method comprises administering to the subject a composition comprising the virus like vesicles (VLVs) produced by the VLV producing vector of the disclosure, wherein the VLV producing vector comprises DNA encoding a SARS-CoV-2 gene or fragment thereof. EExpression of the SARS-CoV-2 gene or fragment thereof induces an immune response to the SARS-CoV-2 protein or fragment thereof in the subject, which is sufficient to diminish the risk that the subject will develop a disease associated with SARS-CoV-2 infection. In certain embodiments, the disease is COVID-19.

SARS-CoV-2 antigens useful in the methods of the disclosure include, but are not limited to the full-length spike protein (s protein), the extracellular domain of the spike protein, the receptor binding domain (RBD) of the spike protein, the nucleocapsid protein (n protein), a fragment or domain of the nucleocapsid protein, and any combination of antigens or antigens fragments thereof. In some aspects, combinations of SARS-CoV-2 antigens can be assembled into a fusion protein construct useful to induce immune responses against multiple antigens simultaneously.

In another aspect of the present disclosure contemplates the use of a variant of SARS-CoV-2 wherein the variant comprises one or more mutations in the gene encoding at least one of the SARS-CoV-2 spike protein and nucleocapsid protein. The mutation resulting in at least one amino acid addition, substitution and/or deletion of the mutated SARS-CoV-2 protein.

In certain embodiments, the VLV-SARS-CoV-2 vaccine of the disclosure generates short and long term immune responses useful for treating, ameliorating, and/or clearing an established SARS-CoV-2 infection and providing immunity against future exposure to SARS-CoV-2 virus or variants thereof.

In certain embodiments, the compositions are designated as a first agent. In certain embodiments, the methods comprise administering a first agent and one or more second agents. In certain embodiments, the methods comprise administering a first agent and one or more second agents. In certain embodiments, the first agent and one or more second agents are co-administered. In certain embodiments the first agent and one or more second agents are co-administered sequentially or concomitantly.

In certain embodiments, the one or more second agents are also a compound or composition described herein. In certain embodiments, the one or more second agents are different from a compound or composition described herein. Examples of one or more second agents include, but are not limited to, an anti-inflammatory agent, chemotherapeutic agent, and/or anti-infection agent.

In other related embodiments, the additional therapeutic agent may be an anti-SARS-CoV-2 agent, an anti-viral agent, a chemotherapeutic agent, an antibiotic, an analgesic, a non-steroidal anti-inflammatory (NSAID) agent, an antifungal agent, an antiparasitic agent, an anti-nausea agent, an anti-diarrheal agent, and/or an immunosuppressant agent.

In certain embodiments, the one or more second agents are an anti-SARS-CoV-2 agent. In certain embodiments the anti-SARS-CoV-2 agent can include, but is not limited to, interferon alpha-2b, interferon alpha-2a, and interferon alphacon-1 (pegylated and unpegylated), ribavirin; an SARS-CoV-2 RNA synthesis inhibitor; an SARS-CoV-2 protease inhibitor; an SARS-CoV-2 capsid assembly inhibitor; a second antisense oligomer; an SARS-CoV-2 therapeutic vaccine; an SARS-CoV-2 prophylactic vaccine; or an SARS-CoV-2 antibody therapy (monoclonal or polyclonal).

In certain aspects, the disclosure includes a method of immunizing or vaccinating a subject against a GPC3 associated cancer (e.g. HCC). The method comprises administering to the subject a composition comprising virus like vesicles (VLVs) produced by the VLV producing vectors of the disclosure, wherein VLV producing vector comprises DNA encoding a GPC3 gene or fragment thereof. Expression of the heterogeneous gene induces an immune response to the GPC3 protein or fragment thereof encoded thereby in the subject.

The expression or overexpression of GPC3 has been found to be associated with a number of cancers including, but not limited to, hepatocellular carcinoma (HCC), ovarian clear cell carcinoma, melanoma, squamous cell carcinoma of the lung, hepatoblastoma, nephroblastoma (Wilms tumor), and yolk sac tumor, and some pediatric cancers. Expression of GPC3 in HCC is especially attractive as a therapeutic target given the relative lack of GPC3 expression in surrounding, non-cancerous liver cells and tissues. Thus, in certain embodiments, the compositions and VLVs generated by the vectors of the disclosure which comprise GPC3 are useful for the treatment of HCC.

In one aspect, the disclosure includes a method of generating a memory T cell immune response to the GPC3 protein or fragment thereof in the subject. In certain embodiments, the method comprises administering a composition comprising VLVs produced by the VLV-producing vector of the disclosure to a subject in an amount effective to elicit an immune response in the subject. In certain embodiments, the method comprises administering a second effective amount of the composition of VLVs produced by the VLV producing vector of the disclosure at a second, subsequent time period, wherein T memory cells directed against the GPC3 antigen or fragment thereof are generated in the subject. In this way, the production of memory T cells specific for the GPC3 protein or fragment thereof are sufficient to help treat a GPC3 associated cancer in the subject or to provide immunity against the development of a GPC3 associated cancer.

In another aspect, the disclosure includes a method of generating an adaptive B cell immune response the GPC3 protein or fragment thereof in the subject. In certain embodiments, the method comprises administering a composition comprising VLVs produced by the VLV producing vector of the disclosure to a subject in an amount effective to elicit an immune response in the subject. In certain embodiments, the method comprises administering a second effective amount of the composition of the disclosure at a second.

subsequent time period, wherein B memory cells directed against the GPC3 antigen or fragment thereof are generated in the subject. In this way, the production of memory B cells specific for the GPC3 protein or fragment thereof produce antibodies which are sufficient to help treat an existing GPC3 associated cancer in the subject or to provide immunity against development of a GPC3 associated cancer. In certain embodiments, the GPC3 associated cancer is HCC, ovarian clear cell carcinoma, melanoma, squamous cell carcinoma of the lung, hepatoblastoma, nephroblastoma (Wilms tumor), and yolk sac tumor, or some pediatric cancers. In certain embodiments, the GPC3 associated cancer is HCC.

In another respect, the disclosure includes a method of treating, ameliorating, and/or preventing a disease in a subject in need thereof. In certain embodiments, the method comprises administering to the subject a composition comprising virus like vesicles (VLVs) produced by the VLV producing vector of the disclosure, wherein the VLV producing vector comprises DNA encoding a GPC3 gene or fragment thereof and wherein expression of the GPC3 gene or fragment thereof induces an immune responses against the GPC3 gene or fragment thereof that is of benefit to the subject. In certain embodiments, the disease is cancer associated with GPC3 expression and/or overexpression. In certain embodiments, the disease is hepatocellular carcinoma (HCC).

Additionally, in another aspect, the disclosure includes a method of diminishing the risk that a subject will develop a disease associated with GPC3 expression and/or overexpression. The method comprises administering to the subject a composition comprising the virus like vesicles (VLVs) produced by the VLV producing vector of the disclosure, wherein the VLV producing vector comprises DNA encoding a GPC3 gene or fragment thereof. Expression of the GPC3 gene or fragment thereof induces an immune response to the GPC3 protein or fragment thereof in the subject, which is sufficient to diminish the risk that the subject will develop a disease associated with GPC3 infection. In certain embodiments, the disease is a cancer. In certain embodiments, the cancer is HCC.

GPC3 antigens useful in the methods of the disclosure include, but are not limited to the full-length GPC3 protein, fragments or domains of GPC3 protein, any variants of GPC3 protein known in the art, and any combination thereof.

In another aspect of the present disclosure contemplates the use of a variant of GPC3 wherein the variant comprises one or more mutations in the gene encoding at least one of the GPC3 spike protein and nucleocapsid protein. The mutation resulting in at least one amino acid addition, substitution and/or deletion of the mutated GPC3 protein.

In certain embodiments, the VLV-GPC3 vaccine of the disclosure generates short and long term immune responses useful for treating established GPC3 associated cancers (e.g. HCC). In certain embodiments, the VLV-GPC3 vaccine of the disclosure can be used to prevent the recurrence or relapse of a GPC3 associated cancer (e.g. HCC) which has been treated with another treatment strategy. For example, the VLV-GPC3 vaccine of the disclosure can be administered following resection of a HCC cancer and/or orthotopic transplant of liver tissue in order to reduce the risk of recurrence due to surviving, residual cancer cells.

In certain embodiments, the compositions are designated as a first agent. In certain embodiments, the methods comprise administering a first agent and one or more second agents. In certain embodiments, the methods comprise administering a first agent and one or more second agents. In certain embodiments, the first agent and one or more second agents are co-administered. In certain embodiments the first agent and one or more second agents are co-administered sequentially or concomitantly.

In certain embodiments, the one or more second agents are also a compound or composition described herein. In certain embodiments, the one or more second agents are different from a compound or composition described herein. Examples of one or more second agents include, but are not limited to, an anti-inflammatory agent, chemotherapeutic agent, or immunotherapeutic agent.

In other related embodiments, the additional therapeutic agent may be an anti-GPC3 agent, an anti-viral agent, a chemotherapeutic agent, an antibiotic, an analgesic, a non-steroidal anti-inflammatory (NSAID) agent, an immunotherapeutic agent, an anti-nausea agent, an anti-diarrheal agent, and/or an immunosuppressant agent.

In certain embodiments, the one or more second agents are an anti-inflammatory agent (i.e., an inflammation lowering therapy). In certain embodiments the inflammation lowering therapy can include, but is not limited to, a therapeutic lifestyle change, a steroid, a NSAID or a DMARD. The steroid can be a corticosteroid. The NSAID can be an aspirin, acetaminophen, ibuprofen, naproxen, COX inhibitors, indomethacin and the like. The DMARD can be a TNF inhibitor, purine synthesis inhibitor, calcineurin inhibitor, pyrimidine synthesis inhibitor, a sulfasalazine, methotrexate and the like.

In certain embodiments, the one or more second agents are a chemotherapeutic agent (i.e., a cancer treating agent). Chemotherapeutic agents can include, but are not limited to, daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxy coformycin, 4-hydroxy peroxy cyclophosphoramide, 5-fluorouracil (5-FU), 5-fluorodeoxyuridine (5-FUdR), methotrexate (MTX), colchicine, taxol, vincristine, vinblastine, etoposide, trimetrexate, teniposide, cisplatin, gemcitabine, and/or diethylstilbestrol (DES).

In certain embodiments, the one or more second agents are an anti-infection agent. Examples of anti-infection agents include, but are not limited to, antibiotics, antifungal drugs, and/or antiviral drugs.

In certain embodiments, the one or more second agents are an immunotherapeutic agent. Examples of immunotherapeutic agents include, but are not limited to, antibodies including antibodies against other tumor-expressed antigens and/or regulatory proteins expressed by tumor-specific T cells (checkpoint inhibitor proteins), adoptive cell therapies including chimeric antigen receptor (CAR) expressing T cells, oncolytic viruses, and/or immune system-modulating small molecules.

### Pharmaceutical Compositions and Formulations

In certain embodiments, virus like vesicles produced by the VLV producing SARS-CoV-2 vector of the disclosure are formulated as a pharmaceutical composition.

In certain embodiments, virus like vesicles produced by the VLV producing GPC3 vector of the disclosure are formulated as a pharmaceutical composition.

Such a pharmaceutical composition may be in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In certain embodiments, the pharmaceutical compositions useful for practicing the methods of the disclosure may be administered to deliver a dose of between 10⁵ and 10⁹ PFU per immunization. Multiple doses may be administered daily, weekly, monthly, or any combination determined by the skilled artisan.

In certain embodiments, the pharmaceutical compositions useful for practicing the method of the disclosure may comprise an adjuvant. Suitable adjuvants contemplated by this disclosure include but are not limited to Freund's complete adjuvant, Freund's incomplete adjuvant, Quil A, Detox, ISCOMs or squalene. Pharmaceutical compositions that are useful in the methods of the disclosure may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human subject being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans, other primates and mammals including chickens, pigs, squirrels and woodchucks.

The composition of the disclosure may comprise a preservative from about 0.005% to 2.0% by total weight of the composition. The preservative is used to prevent spoilage in the case of exposure to contaminants in the environment.

### Administration/Dosing

The regimen or schedule of administration of the compositions of the disclosure may affect what constitutes an effective amount. For example, the high titer hybrid-virus vector of the disclosure may be administered to the subject in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present disclosure to a subject, preferably a mammal, more preferably a human, may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated: each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the compositions\ of the disclosure include inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Kits

In some embodiments a kit is provided for treating, preventing, and/or ameliorating an SARS-CoV-2-related disease, disorder or condition, or a symptom thereof, as described herein wherein the kit comprises: a) a compound or compositions as described herein; and optionally b) an additional agent or therapy as described herein. The kit can further include instructions or a label for using the kit to treat, prevent, or ameliorate the SARS-CoV-2-related disease, disorder or condition. In further embodiment, the disclosure is a kit for assays for variant SARS-CoV-2. Such kits may, for example, contain the reagents from PCR or other nucleic acid hybridization technology (microarrays) or reagents for immunologically based detection techniques (ELISpot, ELISA).

In some embodiments a kit is provided for treating, preventing, or ameliorating an GPC3 expression and/or overexpression-related disease, disorder or condition, or a symptom thereof, as described herein wherein the kit comprises: a) a compound or compositions as described herein: and optionally b) an additional agent or therapy as described herein. The kit can further include instructions or a label for using the kit to treat, prevent, or ameliorate the GPC3-related disease, disorder or condition. In further embodiment, the disclosure is a kit for assays for variant GPC3. Such kits may, for example, contain the reagents from PCR or other nucleic acid hybridization technology (microarrays) or reagents for immunologically based detection techniques (ELISpot, ELISA).

### EXAMPLES

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

The materials and methods employed in the experiments disclosed herein are now described.

### Materials and Methods

Cell line and plasmid information. Hamster kidney cells (BHK-21) and Huh7.5 cell line cells were cultured and maintained in Dulbecco's modified Eagle's medium (DMEM) supplemented with 10% (vol./vol.) fetal bovine serum (FBS), penicillin (100 U/ml) and streptomycin (100 mg/ml) at 37° C. in a 5% humidified incubator.

The recombinant plasmids encoding RBD sequence or full length of spike protein were constructed. Primers were used for PCR amplification of RBD sequence of spike as forward primer: 5′-GGCGCGGCGCCGGCGCGCCGCGTTAATTAAAATGTTTGTTTTTCTTGTTTTATTGC -3′ (SEQ ID NO:1) and reverse primer: 5′-GCCGCATGCATCCTGCAGGTGTGTAATGTAATTTGACTCC-3′ (SEQ ID NO:2). Primers were used for PCR amplification of full length of spike as forward primer: 5′-TTAATTAAAATGACAGAATCTATTGTTAGATTTCCTAATATT-3′ (SEQ ID NO:3) and reverse primer: 5′-CCTGCAGGTTTGTTTTTAACCAAATTAGTAGACTTTTT-3′ (SEQ ID NO: 4). Then, the fragments of RBD sequence and full length of spike were cloned into pCMV-SFVT2AG plasmids with the modified cloning sites of PacI and SbfI adding on the 5′ and 3′ ends. Here, the gene of interests are inserted upstream of a ribosomal T2A skipping site and VSV G protein. The T2A site allows for expression of both the protein of interests and VSV G protein from the same promoter and, importantly, ensures readthrough of the RBD sequence or full length of spike protein prior to VLV production.

Transfection and recovery. VLV-RBD and VLV-Full were recovered by transfecting BHK-21 cells with plasmid pCMV-SFVRBDT2AG and pCMV-SFVFullT2AG, respectively. In general. BHK-21 cells were seeded the day before transfection at a density of 3×10⁵ cells per well of a 6-well plate. Next day, mixing Fugene HD transfection reagent (Promega Corporation, WI) and DNA complex thoroughly and adding to the cells. When the cytopathic effects were observed, the supernatants containing recombinant VLV-RBD or VLV-Full were collected by centrifuge at 600 g for 10 min to remove cell debris. VLV-RBD and VLV-Full were then concentrated by centrifugation through a 100-kDa Amicon Ultra filter unit (EMD Millipore Corporation, MA). VLV-RBD and VLV-Full titer determinations were completed by serial dilution on BHK-21 cells and quantified as PFU per ml. Aliquots were stored at −80° C.

Immunostaining. BHK-21 cells were seeded on the glass of the chamber slides (Corning, NY) one day prior to infection with VLV-RBD or VLV-Full at MOI=1. The cells were washed with PBS for twice and fixed with 4% paraformaldehyde for 15 min at nearly 24 h post infection. Permeabilizing cells with 0.1% Triton-X 100 for 5 min, followed by washing three times. The cells were blocked with PBS containing 2% FBS and incubated with 1:200 dilution of mouse monoclonal VSV-G antibodies. After extensive washes, the cells were stained with 1:500 dilution of secondary antibodies. The stained slides were observed using a fluorescence microscope (Keyence, IL).

Animals and immunizations. Six-to eight-week-old C57BL/6 mice were obtained from Jackson Laboratory. All mice were housed at Yale University School of Medicine animal facilities, and all experiments were performed in accordance with Yale Institutional Animal Care and Use Committee-approved procedures.

C57BL/6 mice (n=5) were intraperitoneal injected with VLV-RBD or VLV-Full at 10⁸ PFU on Day 0 and bled on day 28 for isolating serum to detect spike-specific antibody recognition. To optimize the procedures, VLV-Full was also injected at 10⁸ PFU on Day 0 with intramuscular injection (i.m.), intraperitoneal injection (i.p.) and intranasal inoculation.

C57BL/6 mice (n=5) were intramuscularly immunized with Pfizer mRNA vaccine at 10 ng (equivalent amount). 100 ng and 1 μg on days 0 and 14, and bled on day 28 for isolating serum.

Indirect enzyme-linked immunosorbent assay (ELISA). In brief, 96-well plates were coated with 0.2 μg/well purified spike protein (ACRO Biosystems, DE) in PBS at 4° C. overnight and blocked with 5% non-fat dry milk at 37° C. for 1 h. The serum was diluted at various ratio by using PBST (PBS containing 0.05% Tween 20) and added 100 μl to each well, followed by incubating at 37° C. for 1 h. After being washed with PBST, goat anti-mouse IgG/HRP was added and incubated at 37° C. for 1 h. After being washed, the substrate was added to the plate and incubated in the dark for 15 min. Then, the substrate reaction was stopped by adding H₂SO₄ and the absorbance was read at 492 nm. Alternatively, the antibody levels were also expressed as end point titers. The serum obtained from the mice were serially 10-fold diluted from 1:100 to 1:100000, and then tested by plate reader to obtain the corresponding values.

Proliferation assay. Splenocytes isolated from the immunized mice were stained with 5 mM carboxyfluorescein diacetate succinimidyl ester (CFSE) for 15 min at room temperature. After being washed in PBS, the cells were plated in 24-well plates at a concentration of 10⁷ cells/ml in 1 ml DMEM supplemented with 10% FBS. The cells were incubated with or without purified spike protein for 96 h at 37° C. in a 5% CO2 incubator. PE Cy7-conjugated anti-CD4 antibody was used to detect the proliferation of CD4+ T cells by flow cytometry. All stained cells were analyzed by LSR II. Live cells were carefully gated by forward and side scattering. Data were analyzed with FlowJo software version 10 (FlowJo LLC, OR).

Neutralizing assay. Briefly, 1×10⁴ Huh7.5 cells were seeded in 96-well plate. The serum obtained from the mice were serially 2-fold diluted from 1:20 to 1:640, and then mixed with VSVAG-GFP pseudotyped with codon-optimized S and anti-VSV G for 1 h at 37° C. The mixture was transferred to the corresponding well with Huh7.5 cells for incubating 24-48 h. The GFP spots were observed using a fluorescence microscope (Keyence, IL).

Data analysis. Data analysis are shown as mean±SD. All calculations and statistical analyses were performed using Graphpad Prism (GraphPad Software, CA). Differences were considered statistically significant at P<0.05.

The results of the experiments are now described in the following examples.

### Example 1: Development of a SARS-CoV-2 VLV Vaccine

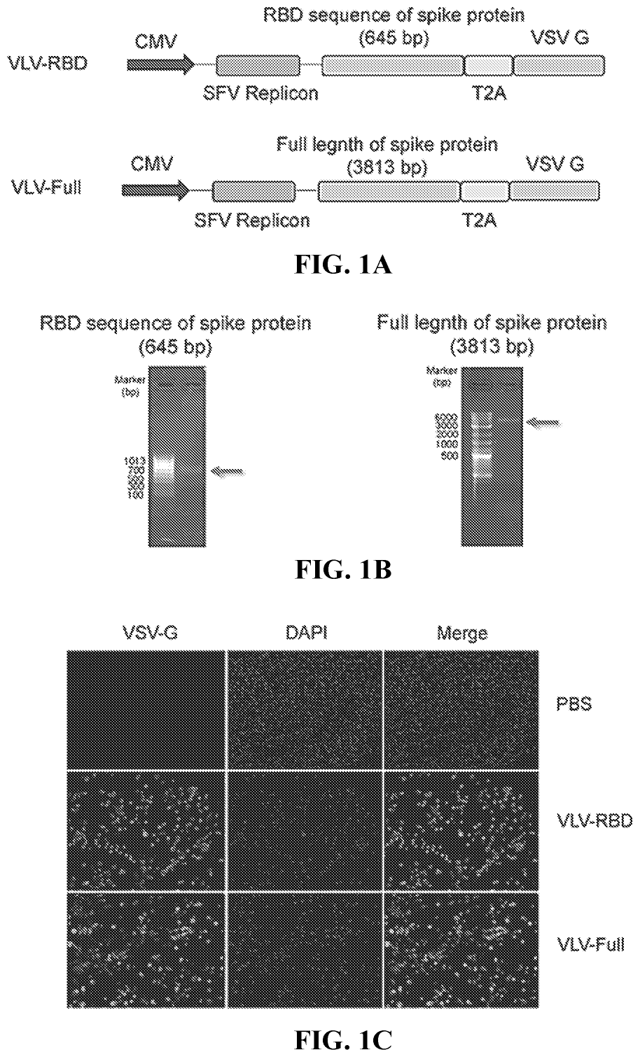

FIG. 1A depicts a diagram of the design of nucleic acid constructs comprising replicating VLVs for expression of SARS-CoV-2 receptor binding domain (RBD) or full length SARS-CoV-2 spike protein. The plasmid constructions contain T2A self-cleaving peptides that allow it to express both antigen and VSV-G protein. FIG. 1B are agarose gels illustrating the amplification of RBD and full-length sequence of SARS-CoV-2 spike protein DNA from B1.1.7 strain. Validation of VSV-G expression in BHK-21 cells after infection with VLV-RBD or VLV-Full (MOI=1) was determined by immunofluorescence at 24 h post infection and is shown in FIG. 1C.

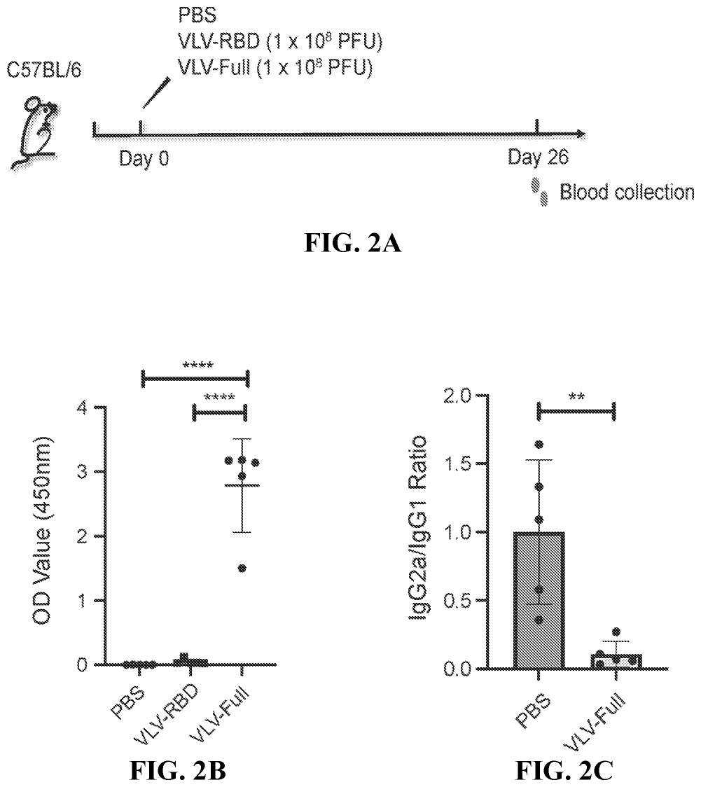

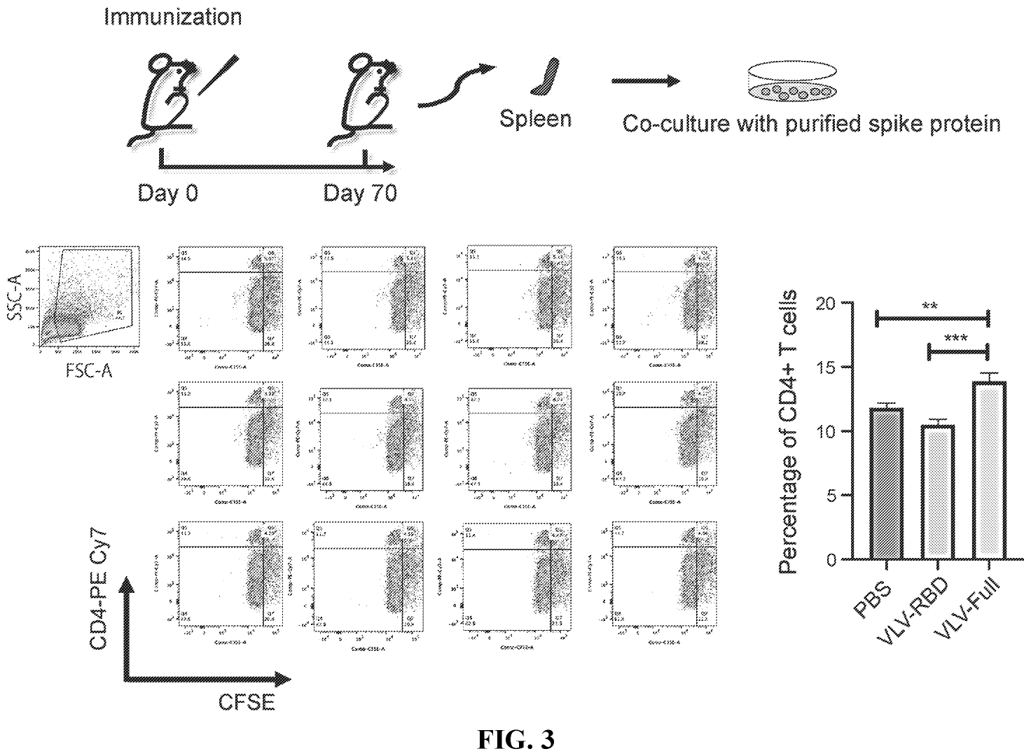

Studies were then conducted to assess anti-spike protein antibody responses in the serum of C57BL/6 mice immunized with PBS, VLV-RBD or VLV-Full. FIG. 2A depicts the experimental procedure for these studies. FIG. 2B illustrates antibody levels, expressed as OD value, in the serum of C57BL/6 mice (n=5) on day 26 after the immunization with PBS, VLV-RBD (1×10⁸ PFU/mice) or VLV-Full (1×10⁸ PFU/mice). FIG. 2C The ratio of IgG2a/IgG1 in the serum of C57BL/6 mice (n=5) on day 26 after the immunization with VLV-Full (1×10⁸ PFU/mice) indicating a robust Th1-type immune response. Bar represents mean±standard deviation (SD). **, P<0.01: ****, P<0.001. Together these data demonstrated the ability of VLV-based vaccines to induce antigen-specific Th1 immune responses.

The effects of purified spike protein on CD4+ T cell proliferation in vaccinated mice were then assessed. Splenocytes isolated from the C57BL/6 mice immunized with PBS, VLV-RBD or VLV-Full on day 0. CFSE-treated splenocytes were incubated with purified spike protein (5 μg/well). After 96 h of incubation, the cells were stained with PE-Cy7 labelled anti-mouse CD4 mAb and evaluated for analyzing the proliferation of CD4+ T cells by flow cytometry. Similar to previous studies, VLV-Full was found to induce a higher percentage of proliferating CD4+ T cells.

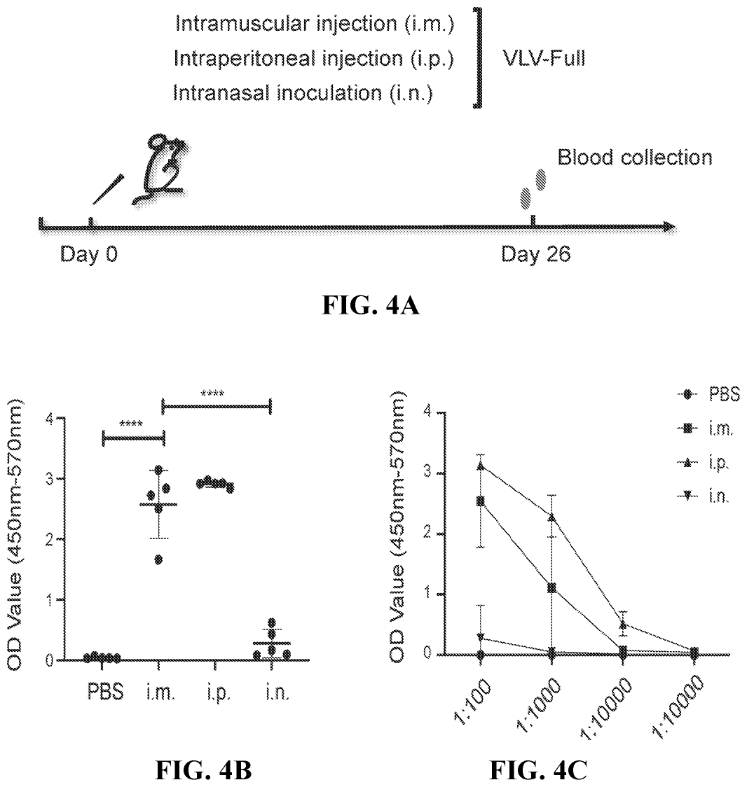

Having established the ability to induce specific anti-SARS-CoV-2 spike protein immune responses, the effects of various injection methods on anti-spike protein immune responses was then assessed. FIG. 4A illustrates the experimental procedure for these studies. Animals were vaccinated with VLV-Full or a PBS control on day 0, and blood was harvested on day 26. FIG. 4B depicts antibody levels, expressed as OD value, in the serum of C57BL/6 mice (n=5) on day 26 after the immunization with VLV-Full (1×10⁸ PFU/mice) using different injection methods, including intramuscular injection (i.m.), intraperitoneal injection (i.p.) or intranasal inoculation (i.n.). Data demonstrated that i.m. and i.p. injection induced equivalent antibody levels, which were significantly higher than i.n. inoculation. FIG. 4C illustrates antibody series dilution in the serum of C57BL/6 mice (n=5) on day 26 after the immunization with VLV-Full (1×10⁸ PFU/mice) by using different injection methods. These data demonstrated that i.p. injection generated modestly high-affinity antibodies than i.m. injection.

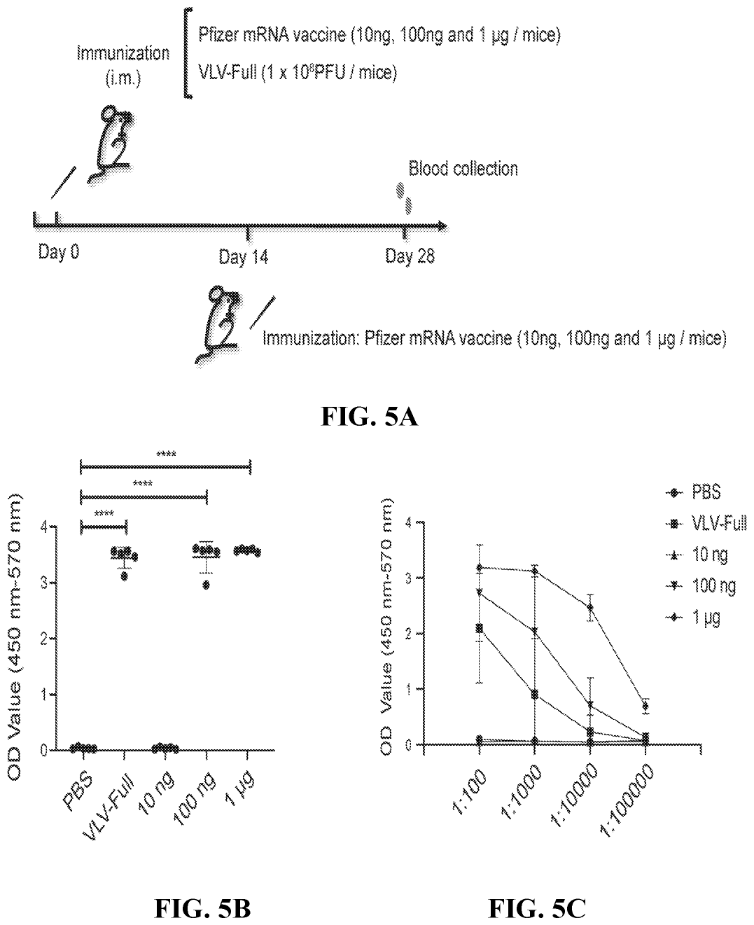

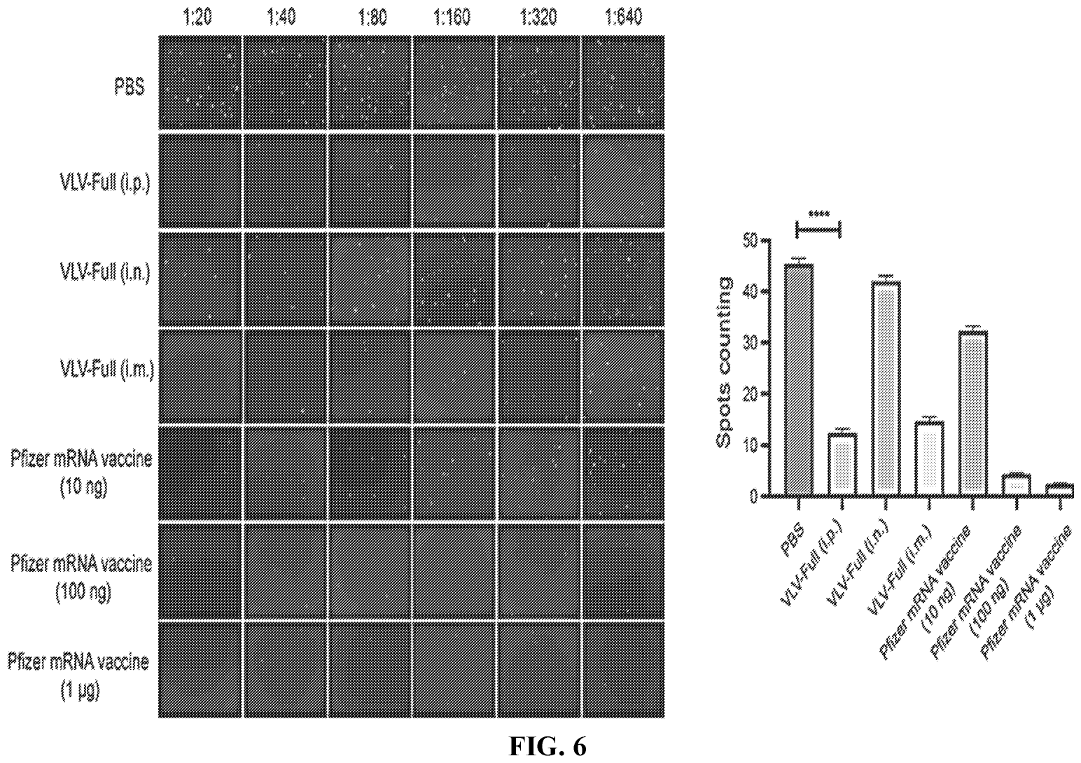

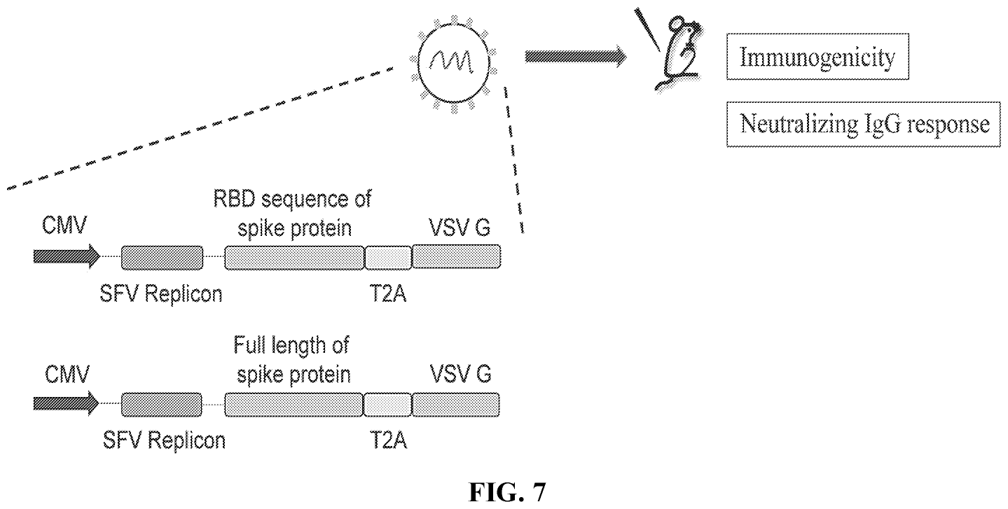

VLV-based vaccines show great promise for clinical use in generating anti-SARS-CoV-2 immune responses. In order to compare the efficacy of VLV-spike protein vaccines vs mRNA-based vaccines, the current clinical gold-standard technology, a series of studies were conducted in which antibody responses in C57BL/6 mice immunized with VLV-Full or Pfizer mRNA vaccine were compared. FIG. 5A illustrates the experimental procedure. FIG. 5B illustrates antibody levels, expressed as OD value, in the serum of C57BL/6 mice (n=5) on day 28 after the immunization with VLV-Full (1×10⁸ PFU/mice) or Pfizer mRNA vaccine administered in different amounts (10 ng, 100 ng and 1 μg). FIG. 5C shows an antibody series dilution in the serum of C57BL/6 mice (n=5) on day 26 after the immunization with VLV-Full (1×10⁸ PFU/mice) or Pfizer mRNA vaccine with different amount (10 ng, 100 ng and 1 μg). In total, these data demonstrated that the VLV-Full vaccine generate equivalent levels of antibodies to 100 ng and 1 μg doses of the Pfizer mRNA vaccine in vivo with a dilution series that most closely resembled the 100 ng mRNA dose, but not significantly worse than the 10 ng mRNA dose except at dilutions above 1:10.000.

Lastly, a neutralization assay was performed in order to demonstrate the functional ability of antibodies generated by VLV vaccination. VSVAG-GFP pseudotyped with spike protein was incubated with series dilutions of serum and VSV-G antibody for 1 h at 37° C. The mixture was then transferred to the corresponding wells with Huh7.5 cells and incubated for 48 h at 37° C. Apparent number of GFP+ spots were counted and showed in the graph. Bar represents mean +standard deviation (SD). ****, P<0.001. These data demonstrated that serum generated by VLV-Full vaccination delivered by i.p. and i.m. resulted in significantly fewer infected cells than PBS controls, i.n. vaccination, and the 10 ng dose of Pfizer mRNA, though not as few as both 100 ng and μg doses of Pfizer mRNA.

### Example 2: Development of a GPC3 VLV

The materials and methods employed in the experiments of this Example are now described.

Cell line and plasmid information. Hamster kidney cells (BHK-21) and mouse HCC Hepa1-6 cell line cells were cultured and maintained in Dulbecco's modified Eagle's medium (DMEM) supplemented with 10% (vol./vol.) fetal bovine serum (FBS), penicillin (100 U/ml) and streptomycin (100 mg/ml) at 37° C. in a 5% humidified incubator.

The recombinant pCMV-SFVGPC3T2AG plasmid encoding GPC3 was constructed. Primers were used for PCR amplification as forward primer: 5′-AGCTAGCTATTAATTAAAATGGCCGGGACCGTGCGCAC-3′ (SEQ ID NO:5) and reverse primer: 5′-AGCTAGCTATCCTGCAGGGTGCACCAGGAAAAAAAAGC-3′ (SEQ ID NO: 6). Then, the fragment of GPC3 was cloned into pCMV-SFVT2AG plasmids with the modified cloning sites of PacI and SbfI adding on the 5′ and 3′ ends, respectively. Here, the gene of interest is inserted upstream of a ribosomal T2A skipping site and VSV G protein. The T2A site allows for expression of both the protein of interest and VSV G protein from the same promoter and, importantly, ensures readthrough of the GPC3 protein prior to VLV production.

Mice. Six-to eight-week-old C57BL/6 mice were obtained from Jackson Laboratory. All mice were housed at Yale University School of Medicine animal facilities.

Transfection and recovery. VLV-GPC3 was recovered by transfecting BHK-21 cells with plasmid pCMV-SFVGPC3T2AG. In general, BHK-21 cells were seeded the day before transfection at a density of 3×10⁵ cells per well of a 6-well plate. Next day, mixing Fugene HD transfection reagent (Promega Corporation, WI) and DNA complex thoroughly and adding to the cells. When the cytopathic effects were observed, the supernatant containing recombinant VLV-GPC3 was collected by centrifuge at 600 g for 10 min to remove cell debris. VLV-GPC3 were then concentrated by centrifugation through a 100-kDa Amicon Ultra filter unit (EMD Millipore Corporation, MA). VLV-GPC3 titer determination was completed by serial dilution on BHK-21 cells and quantified as PFU per ml. Aliquots were stored at −80° C.

Immunostaining. BHK-21 cells were seeded on the glass of the chamber slides (Corning, NY) one day prior to infection with VLV-GPC3 at MOI=1. The cells were washed with PBS for twice and fixed with 4% paraformaldehyde for 15 min at nearly 24 h post infection. Permeabilizing cells with 0.1% Triton-X 100 for 5 min, followed by washing three times. The cells were blocked with PBS containing 2% FBS and incubated with 1:200 dilution of mouse monoclonal VSV-G antibodies. After extensive washes, the cells were stained with 1:500 dilution of secondary antibodies. The stained slides were observed using a fluorescence microscope (Keyence, IL).

Western blot. BHK-21 cells were seeded on 6-well plate one day prior to infection with VLV-GPC3 at MOI=1. The cells were collected and lysed in ice-cold RIPA (Sigma, MO) buffer containing inhibitor (Thermo Fisher Scientific, MA). Then, the lysates were centrifuged at 11000 g for 10 min at 4° C. and the supernatants were collected and quantified using a BCA protein assay kit (Thermo Fisher Scientific, MA). The lysates from the cells were separated by 12% sodium dodecylosulphate-polyacry lamide gel electrophoresis. Gels were blotted using transfer stack (Thermo Fisher Scientific, MA). The membranes were blocked for 1 h at room temperature and probed with anti-GPC3 and anti-GAPDH (Abcam, CT) at 4° C. overnight. After being washed with PBS containing 0.05% Tween 20 three times at 10 min intervals, the membranes were incubated with goat anti-rabbit IgG (Proteintech, IL) or goat anti-mouse IgG (Proteintech. IL) for 1 h at room temperature. After being further washed, the membranes were treated with ECL western blotting substrate (Thermo Fisher Scientific, MA) for visualizing the protein of interests.

Immunization and mouse model establishment. C57BL/6 mice were intraperitoneal injected with VLV-GPC3 at 10⁸ PFU on Day 0 and bled on day 28 for isolating serum to detect GPC3-specific antibody recognition. For therapeutic effects, 1×10⁶ cells mouse HCC Hepa1-6 cell line cells were subcutaneously injected into the right back near the hind leg of the mice on day 28 after immunization. Tumor sizes were measured and calculated every two days. Tumor volume=length×width²×0.5.

For therapeutic effects, 1×10⁶ cells mouse HCC Hepa1-6 cell line cells were subcutaneously injected into the right back near the hind leg of the mice on Day 0. When the tumors were visible to eyes, C57BL/6 mice were injected with VLV-GPC3 at 10⁸ PFU by intramuscular injection (i.m.) or intratumor injection (i.t.). Tumor size was measured and calculated every two days. Tumor volume=length×width²×0.5.

Indirect enzyme-linked immunosorbent assay (ELISA). In brief, 96-well plates were coated with 0.2 μg/well purified GPC3 (Biovision, MA) in PBS at 4° C. overnight and blocked with 5% non-fat dry milk at 37° C. for 1 h. The serum was diluted at various ratio by using PBST (PBS containing 0.05% Tween 20) and added 100 μl to each well, followed by incubating at 37° C. for 1 h. After being washed with PBST, goat anti-mouse IgG/HRP was added and incubated at 37° C. for 1 h. After being washed, the substrate was added to the plate and incubated in the dark for 15 min. Then, the substrate reaction was stopped by adding H₂SO₄ and the absorbance was read at 492 nm. Alternatively, the antibody levels were also expressed as end point titers. The serum obtained from the mice were serially 10-fold diluted from 1:100 to 1:100000, and then the absorbances were tested by plate reader to obtain the corresponding values.

Flow cytometry. Anti-mouse monoclonal antibodies (mAbs) against CD4 (PE-Cy7-conjugated) and CD8a (APC-conjugated) were purchased from Biolegend. The blood samples were lysed with ACK buffer and stained with monoclonal antibodies for 30 min on ice in the dark followed by washing twice with PBS buffer. All stained cells were analyzed by LSR II. Live cells were carefully gated by forward and side scattering. Data were analyzed with FlowJo software version 10 (FlowJo LLC, OR).

ELISPOT. T cell responses were determined using an IFN-γ enzyme-linked immunospot (ELISPOT) set (BD Biosciences, NJ) according to the manufacturer's protocol. Briefly, 96-well plates were used for coating overnight with the purified anti-mouse IFN-γ antibody (1:200). Plates were rinsed and then blocked for 2 h using DMEM supplemented with 10% FBS. Spleen were collected from mice for isolating splenocytes. The splenocytes were passing through 70 μm strainers and lysis with ACK buffer. After washing, cells were suspended in DMEM and seeded at 3×10⁵ cells/well. Purified GPC3 was used to stimulate cells overnight at 37° C. Cells were washed from plates using PBST, and biotinylated anti-mouse IFN-γ antibody (1:250) was added at RT. After washing, HRP (1:100) was added to wells and incubated for 1 h at 25° C. Following the final washes, AEC substrates was added to the wells and allowed to develop at 25° C. for 20 to 40 min. Stop substrate reaction by washing wells with DI water. The plates were allowed to air dry before spot-forming cells (SFC) were counting.

Data analysis. Data analyses are shown as mean±SD. All calculations and statistical analyses were performed using Graphpad Prism (GraphPad Software, CA). Differences were considered statistically significant at P<0.05.

The results of the experiments of this Example are now described.

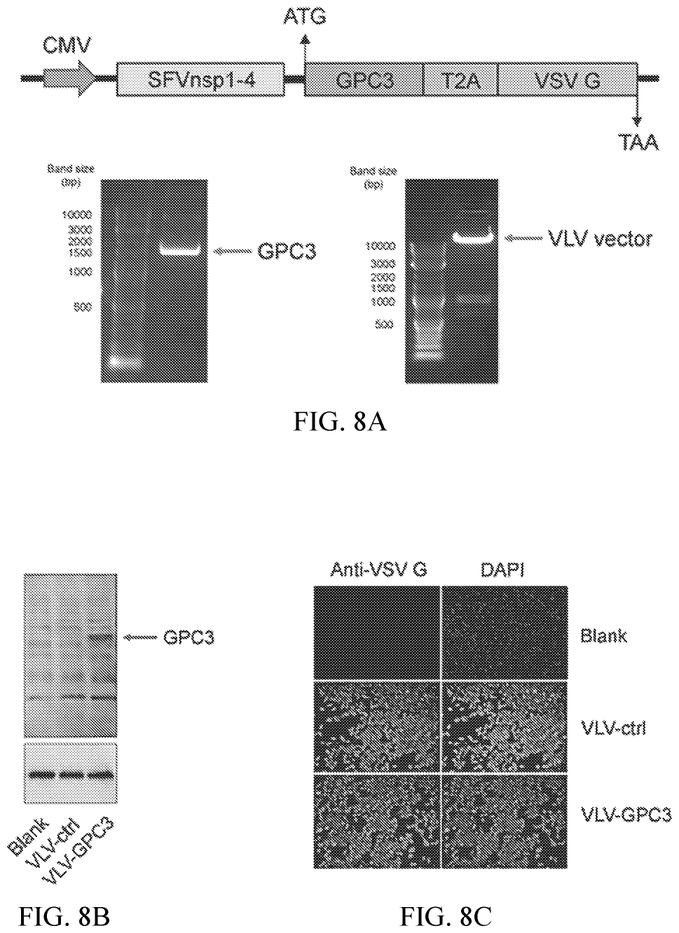

Studies of the current disclosure first sought to establish a virus-like vesicle platform expressing Glypican-3 (GPC3) for use as a vaccine to treat hepatocellular carcinoma (HCC). FIG. 8A illustrates a map of a replicating VLV for expression of GPC3. The plasmid construction contains T2A self-cleaving peptide that allow it to express both GPC3 and VSV-G protein. FIG. 8B demonstrates the evaluation of GPC3 expression in BHK-21 cells after infection with VLV-GPC3 by western blot. VLV-ctrl infected BHK-21 cells were collected and used as negative control. Expression of BHK-21 in vivo was further validated VSV-G expression in BHK-21 cells after infection with VLV-GPC3 (MOI=1) by immunofluorescence at 24 h post infection (FIG. 8C).

### Example 3: In Vivo Use of GPC3 VLVs

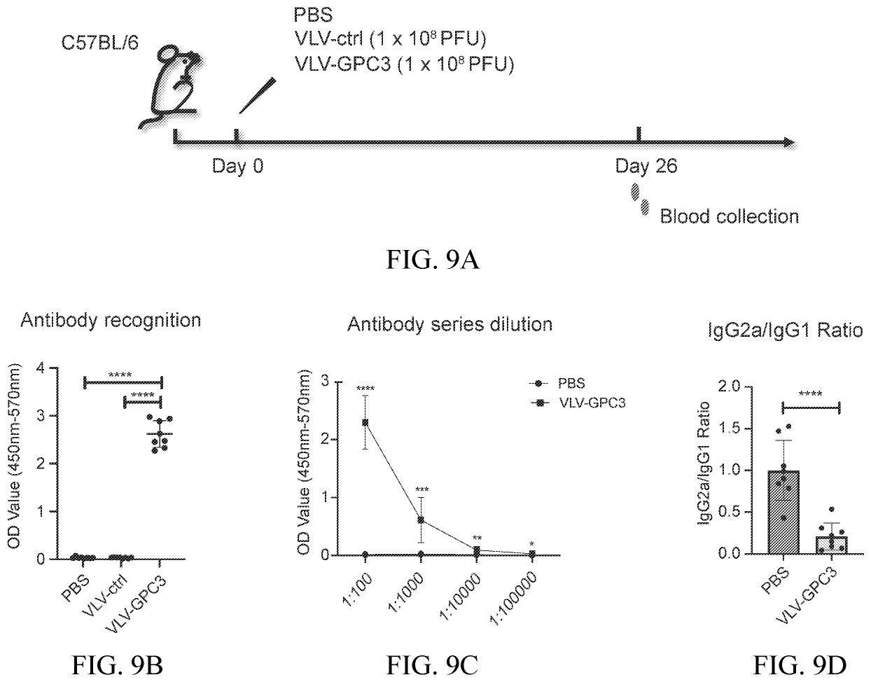

Having demonstrated the ability of GPC3-expressing VLVs to infect and express the payload gene in vitro, studies were then conducted in order to assess whether the VLVs could stimulate anti-GPC immune responses. FIG. 9A illustrates the experimental setup for these studies using 1×10⁸ PFU of either VLV-GPC3, VLV-ctrl, or PBS. After 26 days, peripheral blood was collected from each animal for further analyses. Anti-GPC3 antibody responses in the serum of C57BL/6 mice (n=8) immunized with PBS, VLV-ctrl or VLV-GPC3 were determined and expressed as OD value. These data demonstrated robust anti-GPC3 antibodies generated by animals in the VLV-GPC3 and little detectable antibody in the control groups. An antibody series dilution was then performed using the serum of C57BL/6 mice (n=8) vaccinated mice illustrating the affinity of specific antibodies (FIG. 9C). Lastly, the ratio of IgG2a/IgG1 in the serum of C57BL/6 mice (n=8) was determined on day 26 after the immunization with VLV-GPC3 (1×10⁸ PFU/mice) (FIG. 9D). These data demonstrated that vaccination with VLV-GPC3 could induce significant levels of specific antibodies in the peripheral blood of mice.

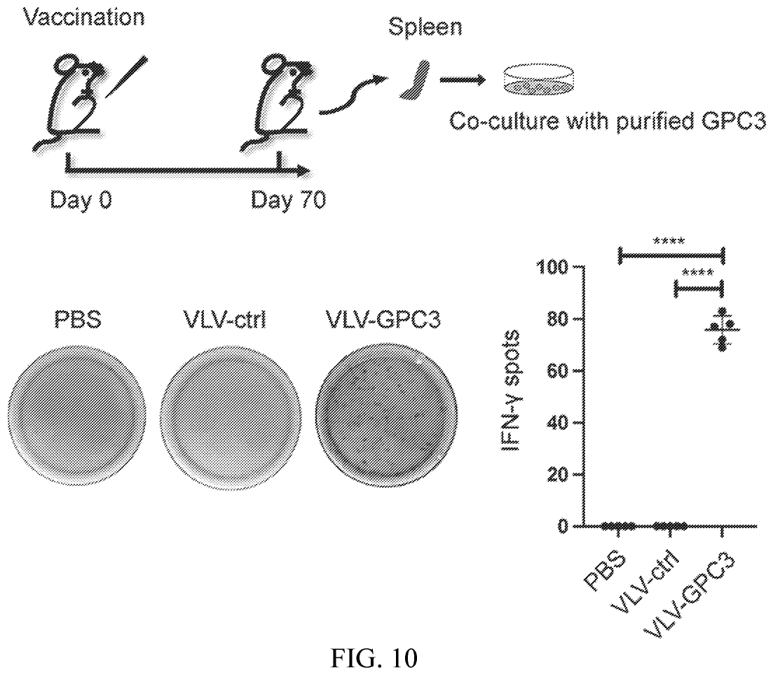

In addition to antibody production, studies also assessed whether antigen-specific memory T cells could be detected in treated mice 70 days following vaccination. These studies utilized ELISPOT analysis to determine IFN-γ secretion in isolated splenocytes incubated with the purified GPC3 protein (FIG. 10). As expected, these studies demonstrated that GPC3-specific T cells could be found in treated mice long after treatment with a single vaccination.

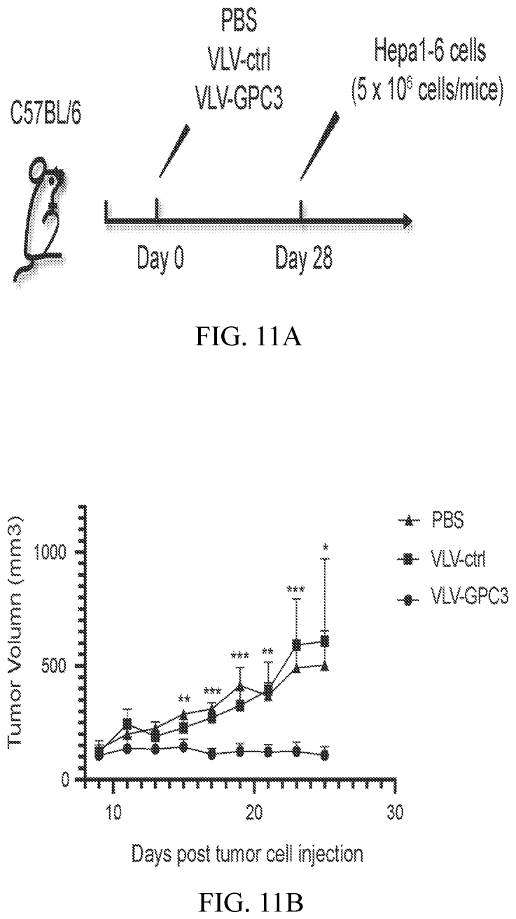

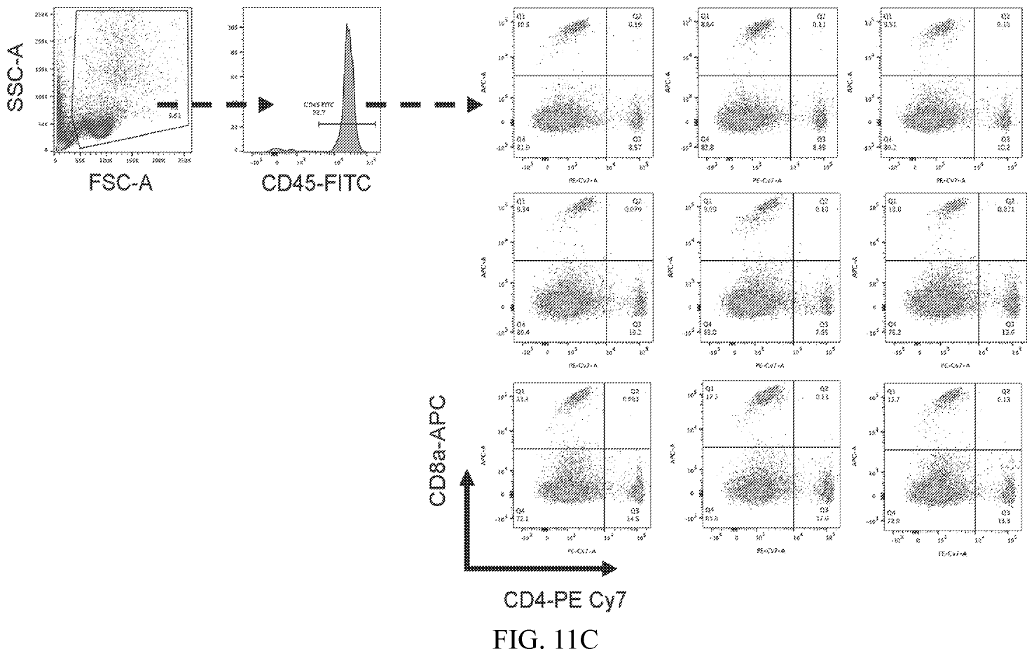

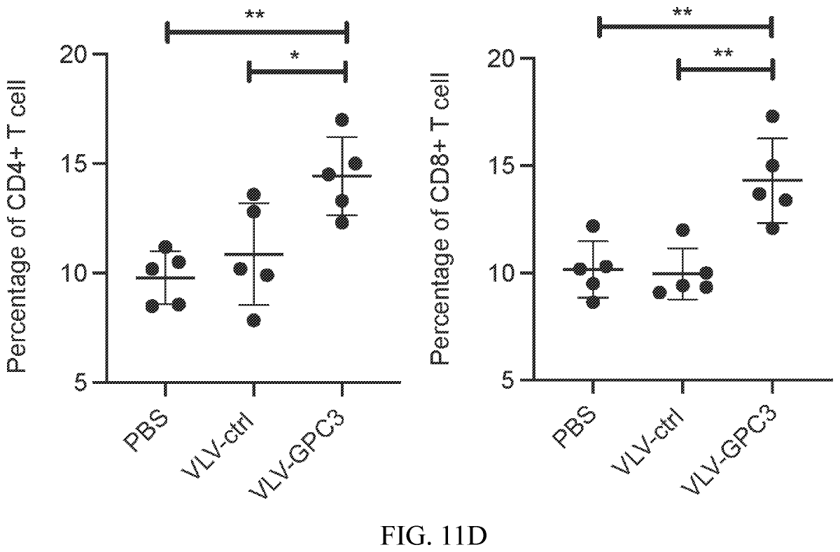

A series of studies were then conducted to assess the ability of anti-GPC3 immune responses primed by the VLV vaccine of the invention to affect the growth of GPC3-expressing tumors in vivo. These studies would be setup in two different treatment settings: preventive, prophylactic vaccination prior to implantation of tumor cells, and a therapeutic setting where vaccination would be used to delay or arrest the growth of an already-established tumor. In the preventive setting, mice were vaccinated with VLV-GPC3 or control twenty-eight days prior to the implantation of tumor cells (illustrated in FIG. 11A). Tumor sizes were measured 3 to 4 times a week and calculated using the equation V=length×width²/2. These studies demonstrated that tumor growth was negligible in GPC3 vaccinated mice while tumors grew out in control-treated mice (FIG. 11B). Flow cytometry was then performed on day 28 to analyze CD4+ T and CD8+ T cell percentage in blood. (FIGS. 11C-11D), data demonstrated higher percentages of CD4+ and CD8+ T cells in VLV-GPC3 vaccinated mice.

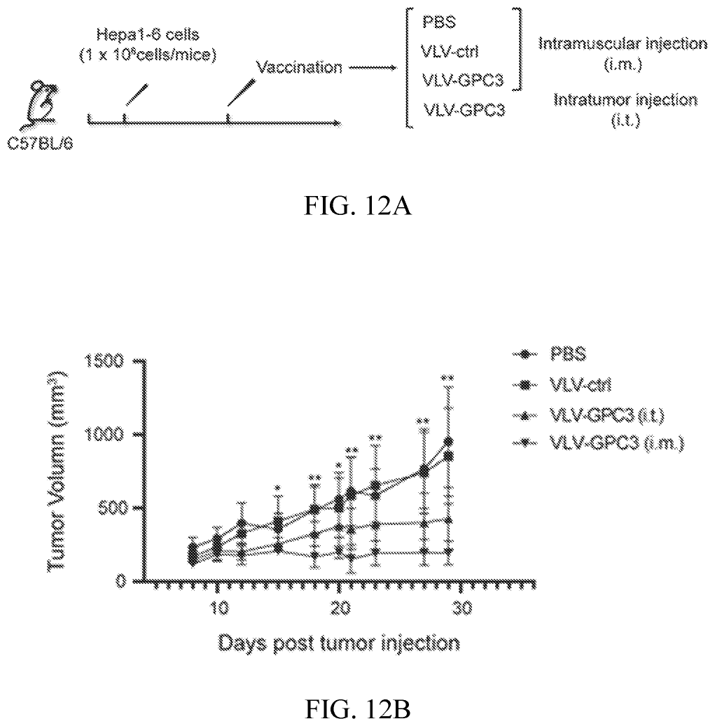

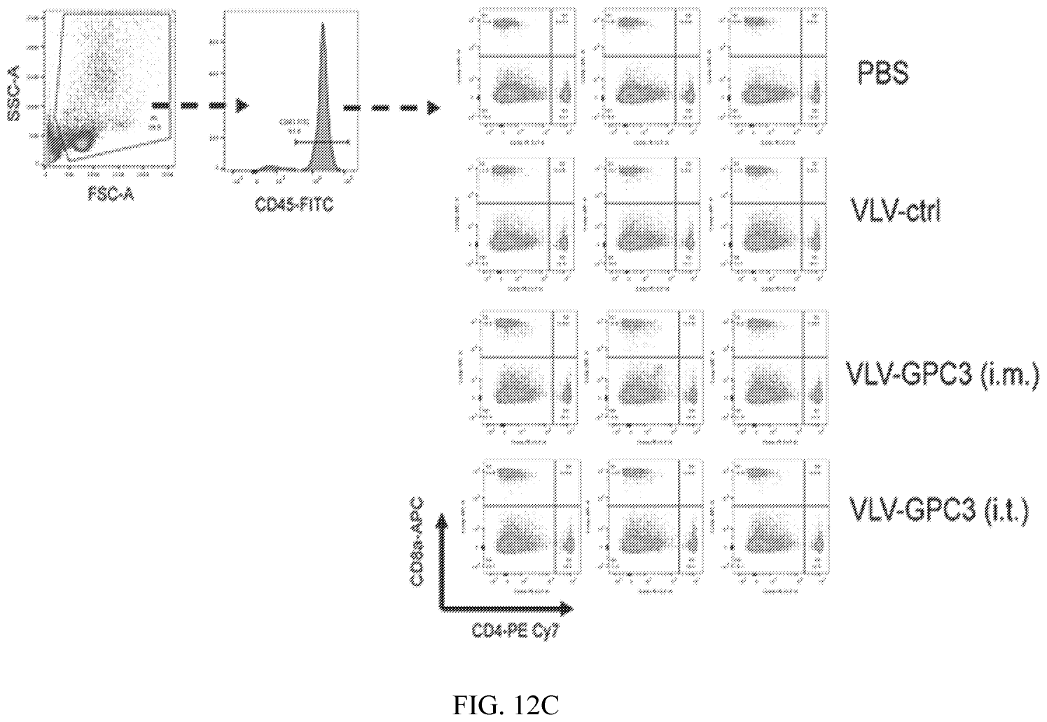

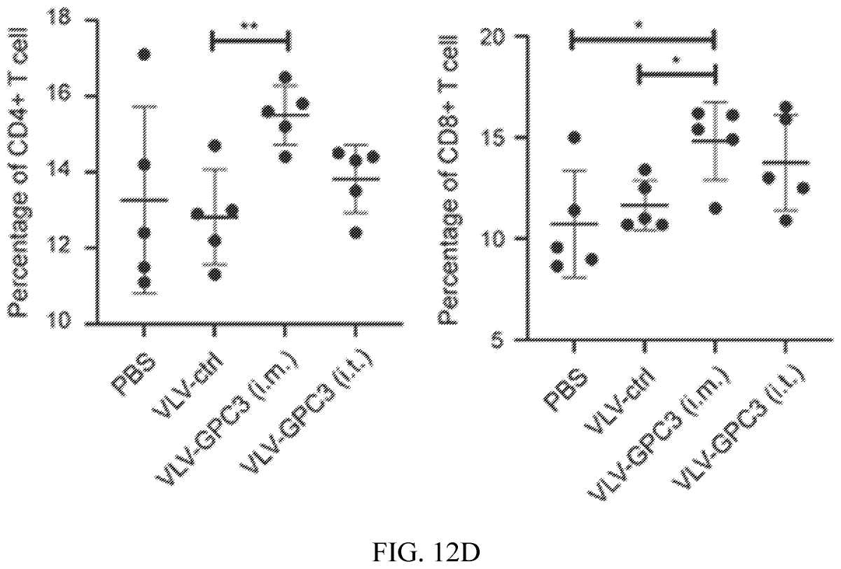

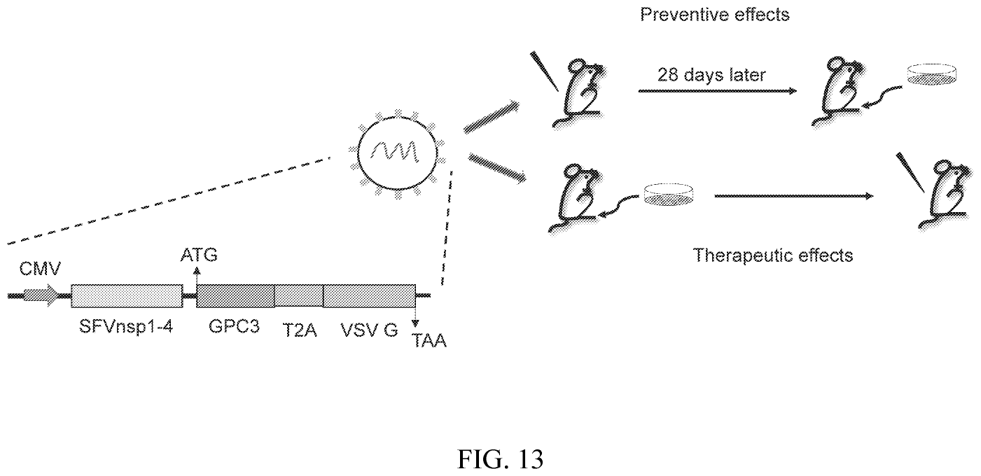

In the therapeutic setting, mice were first injected with 1×10⁶ Hepa1-6 tumor cells prior to vaccination with VLV-GPC3. These studies also examined whether the route of administration could affect tumor growth, with animals receiving either intramuscular (i.m.) or intratumor (i.t.) injections (see diagram in FIG. 12A). Tumor volumes were then measured 3-4 times a week using calipers and tumor volume was calculated using the equation V=length×width²/2. Tumor growth curves demonstrated that GPC3-treated tumors had significantly slower growth than control mice with i.m. treated tumors demonstrating a modestly greater, but statistically insignificant, delay. Similar to the previous study, flow cytometry studies were performed on peripheral blood drawn from mice on day 28 in order to analyze CD4+ T and CD8+ T cell percentages in blood. Here only the GPC3 i.m. treated group was observed to have a significantly higher percentage of CD4+ and CD8+ T cells than the VLV-control group, with only the percentage of CD8+ T cells significantly higher than the PBS control. These studies demonstrated that even control animals generated GPC3-specific T cells, likely a result of some immune recognition of the implanted tumor cells.

Together these studies demonstrated the VLV-based vaccines could be used to generate functional anti-tumor immune responses that could both protect subjects from subsequent antigen-expressing tumors and also treat already-established tumors. Without withing to be bound by theory, these results further suggest the clinical utility of VLV-based vaccines in cancer immunotherapy (illustrated in FIG. 13).

### Enumerated Embodiments

The following enumerated embodiments are provided, the numbering of which is not to be construed as designating levels of importance.

Embodiment 1 provides a virus like vesicle (VLV)-producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a SARS-CoV-2 antigen or fragment thereof, which is operably linked to a 2A DNA encoding a 2A peptide, wherein the 2A DNA is operably linked to a vesicular stomatitis virus (VSV) G DNA encoding a VSV G protein, wherein the SFV non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A. A-11871-G, and T-11978-C, wherein the vector lacks nucleotide sequences which encode SFV structural proteins, further wherein when the vector is propagated in cell culture, titers of at least 10⁷ plaque forming units (pfu) per ml of VLVs are obtained.

Embodiment 2 provides the VLV producing vector of embodiment 1, wherein the promoter sequence comprises a constitutive promoter.

Embodiment 3 provides the VLV producing vector of any one of embodiments 1-2, wherein the promoter sequence comprises the cytomegalovirus immediate early promoter.

Embodiment 4 provides the VLV producing vector of any one of embodiments 1-3, wherein the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof.

Embodiment 5 provides a composition comprising virus like vesicles (VLVs) produced by the VLV producing vector of any one of embodiments 1-4.

Embodiment 6 provides the composition of embodiment 5, wherein the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof.

Embodiment 7 provides the composition of any one of embodiments 5-6, wherein the SARS-CoV-2 antigen is associated with SARS-CoV-2 infection.

Embodiment 8 provides a method of immunizing a subject against SARS-CoV-2 infection, the method comprising administering to the subject a composition comprising at least 10⁷ pfu/ml of the VLVs of any one of embodiments 5-7, wherein expression of the SARS-CoV-2 antigen induces an immune response in the subject.

Embodiment 9 provides the method of embodiment 8, wherein the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof.

Embodiment 10 provides a method of treating, ameliorating, and/or preventing a disease in a subject, the method comprising administering a therapeutically effective amount of the composition of any one of embodiments 5-7 to a subject in need of such treatment.

Embodiment 11 provides the method of embodiment 10, wherein the disease is related to a SARS-CoV-2 infection.

Embodiment 12 provides the method of any one of embodiments 10-11, wherein the disease is COVID-19.

Embodiment 13 provides a method of vaccinating a subject, the method comprising administering to the subject a pharmaceutically acceptable amount of the composition of any one of embodiments 5-7, wherein administration of the composition elicits an immune response in the subject.

Embodiment 14 provides the method of embodiment 13, wherein the composition is a prophylactic vaccine.

Embodiment 15 provides the method of any one of embodiments 13-14, wherein the composition is a therapeutic vaccine.

Embodiment 16 provides the method of any one of embodiments 13-15, wherein the composition is administered in combination with an adjuvant.

Embodiment 17 provides the method of embodiment 16, wherein the adjuvant is selected from the group consisting of Freund's complete adjuvant, Freund's incomplete adjuvant, Quil A, Detox, ISCOMs, and squalene.

Embodiment 18 provides a method of generating a memory T cell immune response to a SARS-CoV-2 antigen or fragment thereof in a subject the method comprising the steps of: (a) administering the composition of any one of embodiments 5-7 to a subject in an amount effective to elicit an immune response in the subject; (b) administering a second effective amount of the composition of any one of embodiments 5-7 at a second, subsequent time period, wherein T memory cells directed against the SARS-CoV-2 antigen or fragment thereof are generated in the subject.

Embodiment 19 provides a method of generating an adaptive B cell immune response to a SARS-CoV-2 antigen or fragment thereof in a subject the method comprising the steps of: (a) administering the composition of any one of embodiments 5-7 to a subject in an amount effective to elicit an immune response in the subject; (b) administering a second effective amount of the composition of any one of embodiments 5-7 at a second, subsequent time period, wherein B memory cells directed against the SARS-CoV-2 antigen or fragment thereof are generated in the subject

Embodiment 20 provides the method of any one of embodiments 8-19, wherein the subject is a mammal.

Embodiment 21 provides the method of embodiment 20, wherein the mammal is a human.

Embodiment 22 provides a VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a SARS-CoV-antigen or fragment thereof, operably linked to a 2A DNA encoding a 2A peptide, which is in turn operably linked to a vesicular stomatitis virus (VSV) G DNA encoding a VSV G protein, wherein the alphavirus non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode alphavirus structural proteins, further wherein when the vector is propagated in cell culture, titers of at least 10⁷ plaque forming units (pfu) per ml of virus like vesicles (VLVs) are obtained.

Embodiment 23 provides a virus like vesicle (VLV) producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a Glypican 3 (GPC3) protein or fragment thereof, operably linked to a 2A DNA encoding a 2A peptide, which is in turn operably linked to a vesicular stomatitis virus (VSV) G DNA encoding a VSV G protein, wherein the SFV non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode SFV structural proteins, further wherein when the vector is propagated in cell culture, titers of at least 10⁷ plaque forming units (pfu) per ml of VLVs are obtained.

Embodiment 24 provides the VLV producing vector of embodiment 23, wherein the promoter sequence comprises a constitutive promoter.

Embodiment 25 provides the VLV producing vector of any one of embodiments 23-24, wherein the promoter sequence comprises the cytomegalovirus immediate early promoter.

Embodiment 26 provides the VLV producing vector of any one of embodiments 23-25, wherein the GPC3 antigen or fragment thereof is GPC3 protein.

Embodiment 27 provides a composition comprising virus like vesicles (VLVs) produced by the VLV producing vector of any one of embodiments 23-26.

Embodiment 28 provides the composition of embodiment 27, wherein the GPC3 antigen or fragment thereof is GPC3 protein.

Embodiment 29 provides the composition of any one of embodiments 27-28, wherein the GPC3 antigen is associated with a GPC3-associated disease.

Embodiment 30 provides the composition of embodiment 29, wherein the GPC-associated disease is a cancer.

Embodiment 31 provides the composition of any one of embodiments 27-30, wherein the cancer is selected from the group consisting of hepatocellular carcinoma (HCC), ovarian clear cell carcinoma, melanoma, squamous cell carcinoma of the lung, hepatoblastoma, nephroblastoma (Wilms tumor), and yolk sac tumor.

Embodiment 32 provides the composition of any one of embodiments 27-31, wherein the cancer is HCC.

Embodiment 33 provides a method of inducing an immune response in a subject against a GPC3 antigen or antigen fragment, the method comprising administering to the subject a composition comprising at least 10⁷ pfu/ml of the VLVs of any one of embodiments 27-32, wherein expression of the GPC3 antigen induces an immune response in the subject.

Embodiment 34 provides the method of embodiment 33, wherein the GPC3 antigen or fragment thereof is GPC3 protein.

Embodiment 35 provides a method of treating, ameliorating, and/or preventing a disease in a subject, the method comprising administering a therapeutically effective amount of the composition of any one of embodiments 27-32 to a subject in need thereof.

Embodiment 36 provides the method of embodiment 35, wherein the disease is related to a GPC3 expression.

Embodiment 37 provides the method of any one of embodiments 35-36, wherein the disease is a cancer.

Embodiment 38 provides the method of embodiment 37, wherein the cancer is selected from the group consisting of hepatocellular carcinoma (HCC), ovarian clear cell carcinoma, melanoma, squamous cell carcinoma of the lung, hepatoblastoma, nephroblastoma (Wilms tumor), and yolk sac tumor.

Embodiment 39 provides the method of any one of embodiments 37-38, wherein the cancer is HCC.

Embodiment 40 provides a method of vaccinating a subject, the method comprising administering to the subject a pharmaceutically acceptable amount of the composition of any one of embodiments 27-32, wherein administration of the composition elicits an immune response in the subject.

Embodiment 41 provides the method of embodiment 40, wherein the composition is a prophylactic vaccine.

Embodiment 42 provides the method of any one of embodiments 40-41, wherein the composition is a therapeutic vaccine.

Embodiment 43 provides the method of any one of embodiments 40-42, wherein the composition is administered in combination with an adjuvant.

Embodiment 44 provides the method of embodiment 43, wherein the adjuvant is selected from the group consisting of Freund's complete adjuvant, Freund's incomplete adjuvant, Quil A, Detox, ISCOMs and squalene.

Embodiment 45 provides a method of generating a memory T cell immune response to a GPC3 antigen or fragment thereof in a subject the method comprising the steps of: (a) administering the composition of any one of embodiments 27-32 to a subject in an amount effective to elicit an immune response in the subject: (b) administering a second effective amount of the composition of any one of embodiments 27-32 at a second, subsequent time period, wherein T memory cells directed against the GPC3 antigen or fragment thereof are generated in the subject.

Embodiment 46 provides a method of generating an adaptive B cell immune response to a GPC3 antigen or fragment thereof in a subject the method comprising the steps of: (a) administering the composition of any one of embodiments 27-32 to a subject in an amount effective to elicit an immune response in the subject: (b) administering a second effective amount of the composition of any one of embodiments 27-32 at a second, subsequent time period, wherein B memory cells directed against the GPC3 antigen or fragment thereof are generated in the subject

Embodiment 47 provides the method of any one of embodiments 33-46, wherein the subject is a mammal.

Embodiment 48 provides the method of embodiment 47, wherein the mammal is a human.

Embodiment 49 provides a VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a GPC3 antigen or fragment thereof, operably linked to a 2A DNA encoding a 2A peptide, which is in turn operably linked to a vesicular stomatitis virus (VSV) G DNA encoding a VSV G protein, wherein the alphavirus non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode alphavirus structural proteins, further wherein when the vector is propagated in cell culture, titers of at least 10⁷ plaque forming units (pfu) per ml of virus like vesicles (VLVs) are obtained.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A virus like vesicle (VLV)-producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a SARS-CoV-2 antigen or fragment thereof, which is operably linked to a 2A DNA encoding a 2A peptide,
wherein the 2A DNA is operably linked to a vesicular stomatitis virus (VSV) G DNA encoding a VSV G protein,
wherein the SFV non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C,
wherein the vector lacks nucleotide sequences which encode SFV structural proteins,
further wherein when the vector is propagated in cell culture, titers of at least 10⁷ plaque forming units (pfu) per ml of VLVs are obtained.

2. The VLV producing vector of claim 1, wherein the promoter sequence comprises a constitutive promoter.

3. The VLV producing vector of claim 2, wherein the promoter sequence comprises the cytomegalovirus immediate early promoter.

4. The VLV producing vector of claim 1, wherein the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof.

5. A composition comprising virus like vesicles (VLVs) produced by the VLV producing vector of claim 1.

6. The composition of claim 5, wherein at least one of the following applies:
(a) the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof;
(b) the SARS-CoV-2 antigen is associated with SARS-CoV-2 infection.

7. (canceled)

8. A method of immunizing a subject against SARS-CoV-2 infection, the method comprising administering to the subject a composition comprising at least 10⁷ pfu/ml of the VLVs of claim 5, wherein expression of the SARS-CoV-2 antigen induces an immune response in the subject;
optionally wherein the SARS-CoV-2 antigen or fragment thereof is selected from the group consisting of SARS-CoV-2 spike protein, SARS-CoV-2 spike protein receptor binding domain (RBD), SARS-CoV-2 nucleocapsid protein, and any combination thereof;
optionally wherein the subject is a mammal, optionally wherein the mammal is a human

9. (canceled)

10. A method of treating, ameliorating, or preventing a disease in a subject, the method comprising administering a therapeutically effective amount of the composition of claim 5 to a subject in need of such treatment,
optionally wherein the disease is related to a SARS-CoV-2 infection, optionally the disease is COVID-19;
optionally wherein the subject is a mammal, optionally wherein the mammal is a human.

11-12. (canceled)

13. A method of vaccinating a subject, the method comprising administering to the subject a pharmaceutically acceptable amount of the composition of claim 5, wherein administration of the composition elicits an immune response in the subject;
optionally wherein the subject is a mammal, optionally wherein the mammal is a human.

14. The method of claim 13, wherein at least one of the following applies:
(a) the composition is a prophylactic vaccine;
(b) the composition is a therapeutic vaccine;
(c) the composition is administered in combination with an adjuvant, optionally wherein the adjuvant is selected from the group consisting of Freund's complete adjuvant, Freund's incomplete adjuvant, Quil A, Detox, ISCOMs, and squalene.

15-17. (canceled)

18. A method of generating a memory T cell immune response to a SARS-CoV-2 antigen or fragment thereof in a subject the method comprising the steps of: (a) administering the composition of claim 5 to a subject in an amount effective to elicit an immune response in the subject; (b) administering a second effective amount of the composition of claim 5 at a second, subsequent time period, wherein T memory cells directed against the SARS-CoV-2 antigen or fragment thereof are generated in the subject;
optionally wherein the subject is a mammal, optionally wherein the mammal is a human.

19. A method of generating an adaptive B cell immune response to a SARS-CoV-2 antigen or fragment thereof in a subject the method comprising the steps of: (a) administering the composition of claim 5 to a subject in an amount effective to elicit an immune response in the subject; (b) administering a second effective amount of the composition of claim 5 at a second, subsequent time period, wherein B memory cells directed against the SARS-CoV-2 antigen or fragment thereof are generated in the subject;
optionally wherein the subject is a mammal, optionally wherein the mammal is a human.

20-21. (canceled)

22. A VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a SARS-CoV-antigen or fragment thereof, operably linked to a 2A DNA encoding a 2A peptide, which is in turn operably linked to a vesicular stomatitis virus (VSV) G DNA encoding a VSV G protein, wherein the alphavirus non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode alphavirus structural proteins, further wherein when the vector is propagated in cell culture, titers of at least 10⁷ plaque forming units (pfu) per ml of virus like vesicles (VLVs) are obtained.

23. A virus like vesicle (VLV) producing vector comprising a DNA sequence comprising a promoter sequence operably linked to a DNA sequence encoding Semliki Forest virus (SFV) non-structural protein nucleotide sequences, operably linked to an SFV subgenomic RNA promoter, operably linked to DNA encoding a Glypican 3 (GPC3) protein or fragment thereof, operably linked to a 2A DNA encoding a 2A peptide, which is in turn operably linked to a vesicular stomatitis virus (VSV) G DNA encoding a VSV G protein, wherein the SFV non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode SFV structural proteins, further wherein when the vector is propagated in cell culture, titers of at least 10⁷ plaque forming units (pfu) per ml of VLVs are obtained.

24. The VLV producing vector of claim 23, wherein the promoter sequence comprises a constitutive promoter.

25. The VLV producing vector of claim 24, wherein the promoter sequence comprises the cytomegalovirus immediate early promoter.

26. The VLV producing vector of claim 23, wherein the GPC3 antigen or fragment thereof is GPC3 protein.

27. A composition comprising virus like vesicles (VLVs) produced by the VLV producing vector of claim 23.

28. The composition of claim 27, wherein
the GPC3 antigen or fragment thereof is GPC3 protein or
the GPC3 antigen is associated with a GPC3-associated disease.

29. (canceled)

30. The composition of claim 28, wherein the GPC-associated disease is a cancer.

31. The composition of claim 30, wherein the cancer is selected from the group consisting of hepatocellular carcinoma (HCC), ovarian clear cell carcinoma, melanoma, squamous cell carcinoma of the lung, hepatoblastoma, nephroblastoma (Wilms tumor), and yolk sac tumor.

32. (canceled)

33. A method of inducing an immune response in a subject against a GPC3 antigen or antigen fragment, the method comprising administering to the subject a composition comprising at least 10⁷ pfu/ml of the VLVs of claim 27, wherein expression of the GPC3 antigen induces an immune response in the subject,
optionally wherein the GPC3 antigen or fragment thereof is GPC3 protein;
optionally wherein the subject is a mammal, optionally wherein the mammal is a human.

34. (canceled)

35. A method of treating, ameliorating, and/or preventing a disease in a subject, the method comprising administering a therapeutically effective amount of the composition of claim 27 to a subject in need thereof;
optionally wherein the subject is a mammal, optionally wherein the mammal is a human.

36. The method of claim 35, wherein at least one of the following applies:
the disease is related to a GPC3 expression; or
the disease is cancer.

37. (canceled)

38. The method of claim 36, wherein the cancer is selected from the group consisting of hepatocellular carcinoma (HCC), ovarian clear cell carcinoma, melanoma, squamous cell carcinoma of the lung, hepatoblastoma, nephroblastoma (Wilms tumor), and yolk sac tumor.

39. (canceled)

40. A method of vaccinating a subject, the method comprising administering to the subject a pharmaceutically acceptable amount of the composition of claim 27, wherein administration of the composition elicits an immune response in the subject;
optionally wherein the subject is a mammal, optionally wherein the mammal is a human.

41. The method of claim 40, wherein at least one of the following applies:
(a) the composition is a prophylactic vaccine;
(b) the composition is a therapeutic vaccine;
(c) the composition is administered in combination with an adjuvant, optionally wherein the adjuvant is selected from the group consisting of Freund's complete adjuvant, Freund's incomplete adjuvant, Quil A, Detox, ISCOMs and squalene.

42-44. (canceled)

45. A method of generating a memory T cell immune response to a GPC3 antigen or fragment thereof in a subject the method comprising the steps of: (a) administering the composition of claim 27 to a subject in an amount effective to elicit an immune response in the subject; (b) administering a second effective amount of the composition of claim 27 at a second, subsequent time period, wherein T memory cells directed against the GPC3 antigen or fragment thereof are generated in the subject; optionally wherein the subject is a mammal, optionally wherein the mammal is a human.

46. A method of generating an adaptive B cell immune response to a GPC3 antigen or fragment thereof in a subject the method comprising the steps of: (a) administering the composition of claim 27 to a subject in an amount effective to elicit an immune response in the subject; (b) administering a second effective amount of the composition of claim 27 at a second, subsequent time period, wherein B memory cells directed against the GPC3 antigen or fragment thereof are generated in the subject; optionally wherein the subject is a mammal, optionally wherein the mammal is a human.

47-48. (canceled)

49. A VLV producing vector comprising a DNA sequence comprising a promoter sequence operably linked to a DNA sequence encoding alphavirus non-structural protein nucleotide sequences, operably linked to an alphavirus subgenomic RNA promoter, operably linked to DNA encoding a GPC3 antigen or fragment thereof, operably linked to a 2A DNA encoding a 2A peptide, which is in turn operably linked to a vesicular stomatitis virus (VSV) G DNA encoding a VSV G protein, wherein the alphavirus non-structural protein nucleotide sequences comprise at least two of the mutations selected from the group consisting of G-4700-A, A-5424-G, G-5434-A, T-5825-C, T-5930-C, A-6047-G, G-6783-A, G-6963-A, G-7834-A, T-8859-A, T-8864-C, G-9211-A, A-10427-G, G-11560-A, A-11871-G, and T-11978-C, wherein the vector lacks nucleotide sequences which encode alphavirus structural proteins, further wherein when the vector is propagated in cell culture, titers of at least 10⁷ plaque forming units (pfu) per ml of virus like vesicles (VLVs) are obtained.


SEQUENCE LISTING (6 sequences)

SEQ ID NO: 1 - DNA, 56 bp, synthetic construct
  source 1..56: mol_type = other DNA
ggcgcggcgccggcgcgccgcgttaattaaaatgtttgtttttcttgttttattgc

SEQ ID NO: 2 - DNA, 40 bp, synthetic construct
  source 1..40: mol_type = other DNA
gccgcatgcatcctgcaggtgtgtaatgtaatttgactcc

SEQ ID NO: 3 - DNA, 42 bp, synthetic construct
  source 1..42: mol_type = other DNA
ttaattaaaatgacagaatctattgttagatttcctaatatt

SEQ ID NO: 4 - DNA, 38 bp, synthetic construct
  source 1..38: mol_type = other DNA
cctgcaggtttgtttttaaccaaattagtagacttttt

SEQ ID NO: 5 - DNA, 38 bp, synthetic construct
  source 1..38: mol_type = other DNA
agctagctattaattaaaatggccgggaccgtgcgcac

SEQ ID NO: 6 - DNA, 38 bp, synthetic construct
  source 1..38: mol_type = other DNA
agctagctatcctgcagggtgcaccaggaaaaaaaagc